(19) United States
(12) Patent Application Publication
(10) Pub. No.: US 2024/0207190 A1
(43) Pub. Date: Jun. 27, 2024

(54) FUCOIDAN-FUNCTIONALIZED POLYSACCHARIDE PARTICLES WITH T-PA FOR TARGETED THROMBOLYTIC THERAPY

(71) Applicants: INSERM (INSTITUT NATIONAL DE LA SANTÉ ET DE LA RECHERCHE MÉDICALE), Paris (FR); UNIVERSITÉ PARIS XIII PARIS-NORD, Villetaneuse (FR); UNIVERSITÉ PARIS CITÉ, Paris (FR)

(72) Inventors: Cédric CHAUVIERRE, Paris (FR); Didier TOURNEUR, Paris (FR)

(21) Appl. No.: 18/001,184

(22) PCT Filed: Jun. 8, 2021
(86) PCT No.: PCT/EP2021/065225

§ 371 Date: Dec. 8, 2022

(30) Foreign Application Priority Data
Jun. 9, 2020 (EP) 20305615.5

(51) Int. Cl. A61K 9/16; A61K 38/48; A61P 7/02

(52) U.S. Cl. CPC A61K 9/1652; A61K 9/1694; A61K 38/482; A61P 7/02; C12Y 304/21068

(57) ABSTRACT

There is a dire need for innovative nanomedicine-based solutions for safe and efficient thrombolysis with a non-toxic, biocompatible, and biodegradable thrombus-targeted carrier. In the present invention, polysaccharide hydrogel submicroparticles with remarkable biocompatibility were elaborated by the inverse miniemulsion/crosslinking method. They were functionalized with a fucoidan which has a nanomolar affinity for the P-selectin overexpressed on activated platelets and endothelial cells in vascular diseases. Surprisingly, the inventors show that rtPA (i.e. Alteplase) can be loaded onto the submicroparticles by adsorption, and its amidolytic and fibrinolytic activities were maintained in vitro and in vivo. Thrombus targeting potential of these particles was validated in microfluidic assay under arterial and venous blood shear rates on recombinant P-selectin and activated platelet aggregates. The thrombolytic efficacy of the nanomedicine-based product was tested in a murine model of acute ischemic stroke, revealing faster middle cerebral artery recanalization and reduction in the brain infarct volume and blood-brain barrier permeability post-stroke, evidenced by laser speckle contrast imaging and MRI. Collectively, this proof of concept study demonstrates the potential of these particles for the precise treatment of acute thrombotic events.

### FIELD OF THE INVENTION

The present invention is in the field of medicine and in particular nanomedicine, hematology, and cardiology.

### BACKGROUND OF THE INVENTION

Acute thrombotic pathologies such as myocardial infarction, ischemic stroke, and venous thromboembolism remain a major global healthcare challenge contributing to a significant number of deaths and disabilities [1]. Current thrombolytic therapy, the intravenous injection of Plasminogen Activators (PA), is administrated to lyse a vascular occlusion and restore the blood flow in the vessel. The recombinant tissue plasminogen activator (rtPA) is the most commonly applied clot-busting drug in clinics and the only one approved for the treatment of acute ischemic stroke [2]. rtPA is a fibrin-specific serine protease that activates the endogenous proenzyme plasminogen and converts it to the active form plasmin, thus, degrading the thrombus fibrin network. However, systemic delivery of rtPA is limited by a narrow therapeutic window (4.5 h of stroke symptom onset), rapid drug elimination (half-life 4-6 min), and physiological deactivation by its antidotes such as Plasminogen Activator Inhibitors (PAI-1 and PAI-2), posing the risks of deleterious side-effects such as intracranial hemorrhages [3]. Moreover, the rate of acute recanalization after intravenous administration of rtPA is low: only ˜30% of patients experienced full or partial recanalization identified by CT angiography according to the study [4].

Therapeutic strategies that intend to address the challenges of thrombolytic therapy and to boost survival rates remain of great clinical interest. Certainly, novel thrombolytic molecules are being researched in order to increase reperfusion, improve safety, and protect the brain neurovascular unit [5,6]. Apart from that, nanomedical approaches for targeted delivery of thrombolytic agents have been intensively proposed [7]. Korin et al. reported the microaggregates of poly (lactic-co-glycolic acid) (PLGA) nanoparticles (NPs) dissociated into rtPA-bearing nanocompounds when exposed to abnormally high hemodynamic shear stress, typical for the vascular occlusions, that performed effective thrombolysis in several preclinical models [8]. Colasuonno et al. formulated rtPA-loaded discoidal porous nanoconstructs from a mixture of PLGA and polyethylene glycol (PEG) with high thrombolytic potential that was presumably attributed to the erythrocyte-mimicking shape of the NPs and their deformability, leading to efficient circulation profiles and accumulation on the clot [9]. While these systems with a passive targeting succeeded in a promising thrombolytic efficacy in preclinical studies, more recent and advanced examples are formulated with actively targeted nanocarriers.

Active targeting permits drug accumulation specifically at the thrombus site and has the potential to enhance the enzyme penetration into deeply localized thrombi. Apart from the magnetic nanoparticle targeting under an external magnetic field, active blood clot targeting is currently achieved by directing the functionalized NPs towards fibrin or activated platelets (mostly integrin GPIIb/IIIa and less adhesion receptor P-selectin) with antibodies and/or peptides. Notably, a theranostic system for thrombus molecular imaging and targeted therapy was developed by Zhou et al. when rtPA was encapsulated into the Fe₃O₄-based PLGA NPs, and a cyclic arginine-glycine-aspartic acid (cRGD) peptide was grafted onto the chitosan surface to target GPIIb/IIIa on activated platelets [10]. Nevertheless, both antibodies and peptides have their limitations for targeted drug delivery. The immunogenicity, purity, and sufficient circulation time are the main concerns of the application of the antibodies while peptides might suffer from weak binding affinity, metabolic instability and fast renal clearance due to their small sizes, potential immunogenicity, and often a high costs of peptide synthesis [12].

The effective alternative could be the nanoparticle functionalization with fucoidan [13], a naturally-occurring algae-derived sulfated polysaccharide, that allows a strong tropism for the P-selectin overexpression in cardiovascular pathologies [14,15]. Fucoidan emerged as an affordable high-quality targeting ligand to P-selectin, that was prior validated by our group on polysaccharide microparticles with iron oxide for MRI imaging [16], Technetium-99m-radiolabeled polysaccharide microparticles for SPECT imaging [17], polymer microcapsules [18], and polymer microbubbles for ultrasound imaging [19].

This is critical to ensure an excellent safety profile of the designed carrier for targeted thrombolysis in an attempt for future clinical translation. It should be a priority to select biocompatible and fully biodegradable materials with U.S. Food and Drug Administration (FDA)-approval as well as to realize a scalable production of the nanoformulations according to Good Manufacturing Practice (GMP) [20]. Contrary to the popularity of synthetic polymers such as PLGA, the NPs made of the polysaccharides are explored to a lesser degree for thrombolytic therapy. Yet, they benefit from the general advantages of natural polymers: biocompatibility, low cost, and hydrophilicity. Polysaccharide hydrogels, that are crosslinked three-dimensional polymer networks, are capable of absorbing large quantities of water and can effectively load macromolecules [21], including plasminogen activators, with high encapsulation efficiency. Only a few publications reported the nanoformulations with chitosan, a cationic chitin-derived polysaccharide that can form polyelectrolyte complexes with negatively charged molecules [22]. For instance, superior thrombolytic potential in vivo, both by intravenous injection and catheter-driven, was demonstrated on self-assembled chitosan NPs crosslinked with sodium tripolyphosphate and loaded with urokinase [23]. Liao et al. formulated the lumbrokinase-bearing NPs from quaternized derivative of chitosan—N,N,N-Trimethyl Chitosan covalently grafted with cRGD peptide to target GPIIb/IIIa receptors that could accelerate thrombolysis [24].

Dextran, an exocellular bacterial water-soluble polysaccharide, is extensively employed in clinics, in particular in its low molecular weight (40 and 70 kDa), for plasma volume expansion, thrombosis prophylaxis, peripheral blood flow enhancement, artificial tears, etc. [25]. Dextran coating of magnetic NPs is applied to ensure environmental stability and to prolong the blood circulation time [26,27]. Our group has recently demonstrated that rtPA-immobilized core-shell poly(isobutyl cyanoacrylate) NPs, decorated with dextran and fucoidan, effectively augmented thrombolysis in mice [28]. However, to our knowledge, there are no reported exclusively dextran carriers for thrombolytic therapy. Meeting the requirements of biocompatibility, biodegradability, non-immunogenicity, dextran stands out as an attractive polymer to design a hydrogel-based protein delivery system for the thrombolytic application.

### SUMMARY OF THE INVENTION

As defined by the claims, the present invention relates to fucoidan-functionalized polysaccharide particles with t-PA for targeted thrombolytic therapy.

### DETAILED DESCRIPTION OF THE INVENTION

Thrombolytic therapy is an intravenous administration of clot-busting agents for the treatment of life-threatening acute thromboembolic diseases. However, thrombolytics exhibit limited clinical efficacy because of their short plasma half-lives and risks of hemorrhages. There is a dire need for innovative nanomedicine-based solutions for safe and efficient thrombolysis with a non-toxic, biocompatible, and biodegradable thrombus-targeted carrier.

In the present invention, polysaccharide hydrogel submicroparticles with remarkable biocompatibility were elaborated by the inverse miniemulsion/crosslinking method. They were functionalized with a fucoidan which has a nanomolar affinity for the P-selectin overexpressed on activated platelets and endothelial cells in vascular diseases. Surprisingly, the inventors show that rtPA (i.e. Alteplase) can be loaded onto the submicroparticles by adsorption, and its amidolytic and fibrinolytic activities were maintained in vitro and in vivo. Thrombus targeting potential of these particles was validated in microfluidic assay under arterial and venous blood shear rates on recombinant P-selectin and activated platelet aggregates. The thrombolytic efficacy of the nanomedicine-based product was tested in a murine model of acute ischemic stroke, revealing faster middle cerebral artery recanalization and reduction in the brain infarct volume and blood-brain barrier permeability post-stroke, evidenced by laser speckle contrast imaging and MRI. Collectively, this proof of concept study demonstrates the potential of these particles for the precise treatment of acute thrombotic events.

Accordingly, the first object of the invention relates to a cross-linked polysaccharide particle comprising an amount of fucoidan and loaded with an amount of t-PA.

As used herein, the term “particle” refers to polysaccharide composition of the invention having a substantially spherical or ovoid shape. Typically, the particles of the invention have a size from 1 nm to 1,000 nm, preferably from 250 to 900 nm and even more preferably from 500 to 850 nm in size. In some embodiments, the size of the particle is about 708.48±40.00 nm. For most nanoparticles, the size of the nanoparticles is the distance between the two most distant points in the nanoparticle. Nanoparticle size can be determined by different methods such as Dynamic Light Scattering (DLS), Small Angle X-ray Scattering (SAXS), Scanning Mobility Particle Sizer (SMPS), Scanning Electron Microscopy (SEM), Transmission Electron Microscopy (TEM) (Orts-Gil, G., K. Natte, et al. (2011), Journal of Nanoparticle Research 13(4): 1593-1604; Alexandridis, P. and B. Lindman (2000), Amphiphilic Block Copolymers: Self-Assembly and Applications, Elsevier Science; Hunter, R. J. and L. R. White (1987). Foundations of colloid science, Clarendon Press).

As used herein, the term “polysaccharide” refers to a molecule comprising two or more monosaccharide units. The term “saccharide unit” as used herein means one saccharide molecule. A saccharide unit is a monomeric unit of a polysaccharide. The term “saccharide” is inclusive of carbohydrates, such as glucose, fructose or galactose, and derivatives thereof, such as mannuronic acid or guluronic acid.

Typically, the polysaccharide is selected from the group consisting of dextran, pullulan, carboxymethyl dextran, agar, alginic acid, hyaluronic acid, inulin, heparin, chitosan and mixtures thereof. More preferably, the polysaccharide is dextran.

As used herein, the term “dextran” has its general meaning in the art and is understood to refer to an a-D-1,6 glucose-linked glucan with side chains 1-3 linked to the backbone units of the polysaccharide.

One important feature of the invention is that the polysaccharide is not chemically modified. In particular, the polysaccharide (e.g. dextran) is not aminated by a covalent linkage between the reducing end of the said polysaccharide and a chemical group comprising one or more amino groups. An “amino group” (—NH₂) refers to any chemical group with a free (—NH₂) radical, in particular primary amine groups and guanidine groups, and more particularly primary amine groups. In a non-limitative way, an amino group may be selected in the group consisting in lysine, arginine, ornithine, or γ-aminobutyric acid. Therefore, the particles as described in Juenet M, Aid-Launais R, Li B, et al. Thrombolytic therapy based on fucoidan-functionalized polymer nanoparticles targeting P-selectin. Biomaterials. 2018; 156:204-216. doi: 10.1016/j.biomaterials.2017.11.047 are excluded from the scope of the invention.

As used herein, the “cross-linked” is intended to refer to two or more polymer chains of the polysaccharide which have been covalently bonded via a cross-linking agent.

As used herein, the term “cross-linking” refers to the linking of one polysaccharide chain to another one with covalent bonds.

As used herein, the term “cross-linking agent” encompasses any agent able to introduce cross-links between the chains of the polysaccharides of the invention.

As used herein, the term “fucoidan” has its general meaning in the art and refers a type of polysaccharide, which contains substantial percentages of L-fucose and sulfate ester groups, mainly derived from brown seaweed and some other marine invertebrates. Fucoidans are indeed generally made of a linear backbone built up of α-1,3-L Fucose or alternating α-1,3-L Fucose, α-1,4-L Fucose, or α-1,2-L Fucose which can be present in the backbone branching. Sulfate groups occupy the C-2 and/or C-3 or C-4 of fucose. In some embodiments, fucoidans are α-1,2- or α-1,3-linked L-fucose polymers that are mainly sulfated on position 4 and position 2 or 3 following the glycosidic linkage. However, besides fucose and sulfate residues, fucoidans also contain other monosaccharides (e.g., mannose, galactose, glucose, xylose, etc.) and uronic acid groups. It is known in the art that the structure of fucoidans from different brown algae varies from species to species. When fucoidans contain uronic acid (UA) and other hexoses, the structure of said fucoidans may be built around a polysulfated poly-L fucose linear backbone bearing substituents selected in a group consisting of: uronic acid, an hexose (1 unit), a sulfate group, and an acetyl group. As an example, the schematic widely admitted structure of fucoidan extracted from the brown seaweed Ascophyllum nodosum is given in Berteau & Mulloy or Pomin & Mourao (O. Berteau and B. Mulloy, 2003, Glycobiology, 13(6) 29-40, DOI: 10.1093/glycob/cwg058; V. Pomin and PAS Mourao, 2008, Glycobiology 18(12) 1016-1027, review, DOI:10.1093/glycob/cwn085). In some embodiments, a fucoidan can be composed of a repeating unit of formula (I):

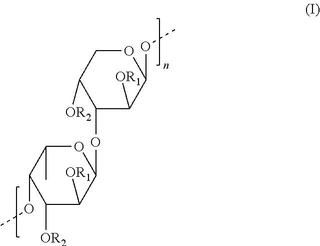

wherein
   R₁ and R₂ mean, one independently from the other: H, a sulfate group, an acetyl group, an hexose and/or uronic acid,
   n is equal or superior to 1.

In some embodiments, the fucoidan suitable for the invention is obtained from seaweed, and in particular brown seaweed (B. Li et al, Molecules, 2008, 13: 1671-1695; M. Kusaykin et al, Biotechnol. J., 2008, 3: 904-915).

Variant forms of fucoidans have also been found in marine animal species, including the sea cucumber. Thus, compared to other sulfated polysaccharides, fucoidans are widely available from various kinds of cheap sources, and easily obtained using methods of extraction known in the art (C. Colliec et al, Phytochemistry, 1994, 35(3):697-700). These methods of extraction generally yield fucoidans with molecular weights in the 70-800 kDa range. Processes have also been developed to depolymerize high molecular weight fucoidans in low molecular weight fucoidans, e.g., lower than about 20 kDa (EP 0403 377B, U.S. Pat. No. 5,321,133), or lower than about 10 kDa (EP 0 846 129 B; U.S. Pat. No. 6,028,191; A. Nardella et al, Carbohydr. Res., 1996, 289: 201-208).

In some embodiments, fucoidans can be obtained commercially from the following companies: Fucoidan from Sigma-Aldrich company (USA): Crude fucoidan (from Fucus vesiculosus) ref F5631, CAS Number 9072-19-9. MM=20,000-200,000 g/mol; Fucoidan from Algues-et-Mer company (France): Asphyscient® (from Ascophyllum nodosum), on request, MM=5,000-10,000 g/mol; Fucoidan from Kraeber GmbH (Germany): on request, LMWF, 8,500 g/mol, HMWF, 600,000 g/mol, from different brown algae.

In some embodiments, the structure of fucoidans can also be chemically modified. For example, methods have been developed to increase the percentage of sulfate groups of fucoidans in order to obtain oversulfated fucoidans or oversulfated fucoidan fragments (T. Nishino et al, Carbohydr. Res., 1992, 229: 355-362; S. Soeda et al, Thromb. Res., 1993, 72: 247-256). According to a particular embodiment, the fucoidan is polysulfated. According to a more particular embodiment, said polysulfated fucoidan has a sulfate-to-sugar ratio superior to 1, in particular superior to 1.2, preferably superior or equal to 1.9.

In some embodiments, the fucoidans can be of high molecular weight or low molecular weight.

As used herein, the term “molecular weight” relates to the average molecular weight, or Mw. A “low molecular weight fucoidan” relates to any fucoidan with an average molecular weight equal or lower than 20,000 Da, in particular within a range between 2,000 and 20,000 Da. A “high molecular weight fucoidan” relates to any fucoidan with an average molecular weight superior to 20,000 Da, in particular within a range between 20,000 and 600,000 Da.

In some embodiments, the fucoidan has an average molecular weight of about 2,000 to about 100,000 Da. In some embodiments, the fucoidan has an average molecular weight of about 20,000 to about 70,000 Da. In some embodiments, the fucoidan has an average molecular weight of about 100,000 to about 500,000 Da. In some embodiments, the fucoidan has an average molecular weight which is lower than 100,000 Da, and preferably lower than 20,000 Da, for instance between 2,000 and 20,000 Da. In some embodiments, the fucoidan has an average molecular weight ranging from 2,000 Da to 1,5000 Da. In some embodiments, the fucoidan is chosen among low molecular weight fucoidans, such as the ones described in WO2010116209.

As used herein, the term “t-PA” has its general meaning in the art and refers to tissue-type plasminogen activator. The term includes native t-PA and recombinant t-PA, as well as modified forms of t-PA that retain the enzymatic or fibrinolytic activities of native t-PA. The enzymatic activity of t-PA can be measured by assessing the ability of the molecule to convert plasminogen to plasmin. The fibrinolytic activity of t-PA may be determined by any in vitro clot lysis activity known in the art. Recombinant t-PA has been described extensively in the prior art and is known to the person of skill. t-PA is commercially available as alteplase (Activase® or Actilyse®). Modified forms of t-PA (“modified t-PA”) have been characterized and are known to those skilled in the art. Modified t-PAs include, but are not limited to, variants having deleted or substituted amino acids or domains, variants conjugated to or fused with other molecules, and variants having chemical modifications, such as modified glycosylation. Several modified t-PAs have been described in PCT Publication No. WO93/24635; EP 352,119; EP382174.

In some embodiments, the cross-linked polysaccharide particle of the invention is obtainable by the method that comprises the following steps:
a) preparing an alkaline aqueous solution comprising an amount of at least one polysaccharide, the amount of fucoidan and an amount of a cross linking agent;
b) dispersing said alkaline aqueous solution into a hydrophobic phase in order to obtain w/o emulsion; and
c) transforming the w/o emulsion into particle by placing said w/o emulsion at a temperature from about 4° C. to about 80° C. for a sufficient time to allow the cross-linking of said amount of polysaccharide and fucoidan,
d) loading the amount of t-PA into the particles obtained at step c).

As used herein, the term “alkaline solution” refers to a solution having a pH strictly superior to 7.

As used herein, the term “aqueous solution” refers to a solution in which the solvent is water.

In some embodiments, the cross-linking agent is selected from the group consisting of trisodium trimetaphosphate (STMP), phosphorus oxychloride (POCI3), epichlorohydrin, formaldehydes, carbodiimides, and glutaraldehydes. Preferably, said cross-linking agent is STMP. Typically, the weight ratio of the polysaccharide to the cross-linking agent is in the range from 15:1 to 1:1, preferably 6:1.

As used herein, “w/o emulsion” or ‘water-in-oil emulsion”, refers to the dispersion of an aqueous phase into a lipophilic phase. The term encompasses stable and non-stable emulsion.

As used herein, the terms “non-aqueous phase”, “lipophilic phase”, “hydrophobic phase”, and “oily phase” may be used in an interchangeable manner.

The skilled artisan is aware of the hydrophobic phases suitable for the purpose of the invention. Non-limiting examples of hydrophobic phases are vegetal oils, such as canola oil, corn oil, cottonseed oil, safflower oil, soybean oil, extra virgin olive oil, sunflower oil, palm oil, MCT oil, and trioleic oil. Preferably, said hydrophobic phase is sunflower oil. Alternatively, said hydrophobic phase is a silicon fluid. Typically, the quantity of hydrophobic phase in the w/o emulsion (volume of lipophilic phase/volume of the water-in-oil emulsion; v/v) represents from about 10% to about 90% v/v, preferably from about 20% to about 80% v/v, preferably from about 50% to about 80% v/v and most preferably about 70% v/v of the w/o emulsion.

Typically, the step b) of dispersing the alkaline aqueous solution into the hydrophobic phase is performed under mechanical stirring. Typically, such a dispersing step is performed during 10 min. Alternatively, the emulsification process can be performed using a high-performance disperser, such as Polytron® Homogenizer.

In some embodiments, step b) of the method of the invention is carried out in presence of a surfactant.

As used herein. “surfactant” or “emulsifier” refers to a compound that lowers the surface tension of a liquid, the interfacial tension between two liquids, or that between a liquid and a solid. In particular a surfactant has an amphiphilic structure which confers thereon a particular affinity for interfaces of water/oil type, thereby giving it the ability to lower the free energy of these interfaces and to stabilize dispersed systems.

In some embodiments, the surfactant is selected from the group consisting of polyglycerol polyricinoleate and PEG-30 dipolyhydroxystearate. Polyglycerol polyricinoleate or PGPR (Palsgaard®4125, Palsgaard®4150, Palsgaard®4110, Palsgaard®4120 or Palsgaard®4175) is a surfactant which has, as hydrophilic group, polyglycerol (preferably consisting of at least 75% of di- and triglycerol and of at most 10% of heptaglycerol) and, as hydrophobic group, interesterified ricinoleiques fatty acids. PEG-30 dipolyhydroxystearate includes Cithrol® DPHS and formerly Arlacel® P135 sold by the company Croda.

In some embodiments, step b) is carried out in presence of an osmotic agent.

As used herein, the term “osmotic agent” refers to a material which creates an osmotic pressure within the oral dosage form which adopted the osmotic system. Upon penetration of fluid into the oral dosage form through semipermeable membrane, osmotic agents are dissolved in the fluid, which creates an osmotic gradient and generates a driving force for the uptake of fluid. Osmotic agents usually are ionic compounds which include but not limited to water-soluble salts, hydrophilic polymers, carbohydrates and water-soluble amino acids. Several osmotic agents are known in the field, which include salts (e.g. NaCl, MgCl2, or KN03), sugars (e.g. sucrose, glucose or fructose), or volatile solutes (e.g. SO2) or certain mixtures thereof.

According to the invention, the particles obtained at step c) have a negative zeta potential.

As used herein, the term “zeta-potential” or “(ζpotential” has its general meaning in the art and refers to the electrical potential that exists across the interface of all solids and liquids, e.g., the potential across the diffuse layer of ions surrounding the particle. Zeta potential can be calculated from electrophoretic mobilities, i.e., the rates at which the particles travel between charged electrodes placed in contact with the substance to be measured, using techniques well known in the art. Typically, said methods include Electrophoretic Light Scattering as described in EXAMPLE.

In particular, the loading of t-PA into the particles obtained at step d) is carried out by mixing the particles with a solution comprising an amount of t-PA. Typically, the loading is carried out by adsorption.

As used herein, the term “adsorption” has its general meaning in the art and refer to adherence of atoms, ions, or molecules of a first substance (e.g. t-PA) the surface of another substance (e.g. cross-linked polysaccharide particle) referred to herein as “the sorbent”. According to the invention the adsorption of t-PA to the surface of the particle does not involve covalent bonds.

Typically, the adsorption is carried out as described in the EXAMPLE. More particularly, the solution comprising the t-PA has a pH below t-PA isoelectric point IP=7.7.

In some embodiments, the method of the invention further comprises a step of calibrating the polysaccharide particles according to their size. After performing said step of calibrating, the person skilled in the art may obtain particles of the desired size.

The size of the polysaccharide particles would be chosen with precaution by the skilled man in regard with the desired use. The size of the polysaccharide particles of the invention is dependent on the characteristics and parameters of the method of preparing such polysaccharide particles. Typically, the size of the polysaccharide particle of the invention may depend on the nature of the polysaccharide, the agitation provided during the process and the distribution of the polysaccharide within the polysaccharide particles. The person skilled in the art may easily adapt and calibrate the particles in order to obtain a desired size. Typically, said adaptation and/or calibration may be performed by the following techniques: sieving or filtration though nylon filter.

In the context of the invention, particles of the invention are “functionalized” by fucoidan meaning that fucoidan is used as a vectorizing agent to confer its specificity/selectivity/affinity property to the selectin. The fucoidan has some degree of affinity for selectins, in particular P-selectin, and that can play a targeting role when they are part of a vectorizing agent. Suitable fucoidan moieties thus include fucoidans that exhibit affinity and specificity for only one of the selectins (i.e., for L-selectin, E-selectin or P-selectin) as well as fucoidans that exhibit affinity and specificity for more than one selectin, including those moieties which can efficiently interact with, bind to or associate with all three selectins. Preferably, the interaction between a selectin and a fucoidan as part of a vectorizing agent is strong enough for at least the time necessary to vectorize t-PA to a thrombus.

As used herein, the terms “binding affinity” and “affinity” are used herein interchangeably and refer to the level of attraction between molecular entities. Affinities can be expressed quantitatively as dissociation constant (K_{D}), or its inverse, the association constant (K_{A}). In some embodiments, a suitable fucoidan interacts with a selectin with a dissociation constant (K_{D}) between about 0.1 nM and about 500 nM, preferably between about 0.5 nM and about 10 nM, more preferably between about 1 nM and about 5 nM.

As used herein, the term “selectin” has its art understood meaning and refers to any member of the family of carbohydrate-binding, calcium-dependent cell adhesion molecules that are constitutively or inductively present on the surface of leukocytes, endothelial cells or platelets. The term “E-selectin”, as used herein, has its art understood meaning and refers to the cell adhesion molecule also known as SELE; CD62E; ELAM; ELAMI; ESEL; or LECAM2 (Genbank Accession Numbers for human E-selectin: NM_000450 (mRNA) and NP_000441 (protein)). As used herein, the term “L-selectin” has its art understood meaning and refers to the cell adhesion molecule also known as SELL; CD62L; LAM-1; LAM1; LECAM1; LNHR; LSEL; LYAM1; Leu-8; Lyam-1; PLNHR; TQ1; or hLHRc (Genbank Accession Numbers for human L-selectin: NM_000655 (mRNA) and NP_000646 (protein)). The term “P-selectin”, as used herein, has its art understood meaning and refers to the cell adhesion molecule also known as a SELP; CD62; CD62P; FLJ45155; GMP140; GRMP; PADGEM; or PSEL (Genbank Accession Numbers for human P-selectin: NM_003005 (mRNA) and NP_002996 (protein)).

A further object of the invention relates to the use of the particles of the invention for therapy (i.e. as a drug).

As used herein, the term “treating”, “treatment” and “therapy” refers to reversing, alleviating, inhibiting the progress of, or preventing the disorder or condition to which such term applies, or one or more symptoms of such disorder or condition. In particular, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In particular, the particles of the invention are particularly suitable for the treatment of thrombotic diseases.

A used herein, the term “thrombotic diseases” and “thrombotic disorders” are diseases and/or disorders which are associated with the appearance, or persistence, of undesirable intravascular thrombus. The term “thrombus” or “blood clot” as used herein refers to a solid or semi-solid mass formed from the constituents of blood within the vascular system that is the product of blood coagulation. There are two components to a thrombus, aggregated platelets that form a platelet plug, and a mesh of cross-linked fibrin protein.

Thrombotic diseases are well-known in the art and can have various causes. They can be primary or acquired diseases. In particular, they can be hereditary, and/or linked to genetic predispositions. Examples of such diseases comprise, for instance, haemophilias, Von Willebrand disease, and other coagulopathies linked to hyper- and hypo-coagulability.

Thrombotic disorders and diseases disclosed herein may, for instance, result in the formation of venous thrombosis such as deep vein thrombosis, portal vein thrombosis, renal vein thrombosis, jugular vein thrombosis, or cerebral venous sinus thrombosis. In some cases, said thrombosis may lead to phlebitis, also referred herein as thrombophlebitis, and sometimes to pulmonary embolisms. It may also involve atrial and ventricular thrombi related to heart arrythmias. They may also result in arterial thrombosis, which is often a consequence of the rupture of an atherosclerotic plaque, in which case it can be also referred as atherothrombosis. An arterial thrombosis may, for instance, lead to a stroke, a myocardial infarction and/or an arterial embolus.

Thus, a further object of the invention relates to a method of treating a thrombotic disease or disorder in a patient in need thereof comprising administering to the patient a therapeutically effective amount a particle of the invention.

As used herein, the expression “therapeutically effective amount” as above described is meant a sufficient amount of the particle for the treatment of the thrombotic disease or disorder. It will be understood, however, that the total daily usage of the compounds and compositions of the invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination with the specific agonist employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

A further object of the invention relates to a pharmaceutical composition comprising an amount of the particles of the invention.

Often, pharmaceutical compositions will be administered by injection. For administration by injection, pharmaceutical compositions of thrombolytic agents may be formulated as sterile aqueous or non-aqueous solutions or alternatively as sterile powders for the extemporaneous preparation of sterile injectable solutions. Such pharmaceutical compositions should be stable under the conditions of manufacture and storage, and must be preserved against the contaminating action of microorganisms such as bacteria and fungi. Pharmaceutically acceptable carriers for administration by injection are solvents or dispersion media such as aqueous solutions (e.g., Hank's solution, alcoholic/aqueous solutions, or saline solutions), and non-aqueous carriers (e.g., propylene glycol, polyethylene glycol, vegetable oil and injectable organic esters such as ethyl oleate). Injectable pharmaceutical compositions may also contain parenteral vehicles (such as sodium chloride and Ringer's dextrose), and/or intravenous vehicles (such as fluid and nutrient replenishers); as well as other conventional, pharmaceutically acceptable, non-toxic excipients and additives including salts, buffers, and preservatives such as antibacterial and antifungal agents (e.g., parabens, chlorobutanol, phenol, sorbic acid, thirmerosal, and the like). Prolonged absorption of the injectable compositions can be brought about by adding agents that can delay absorption (e.g., aluminum monostearate and gelatin). The pH and concentration of the compositions can readily be determined by those skilled in the art. Sterile injectable solutions are prepared by incorporating the active compound(s) and other ingredients in the required amount of an appropriate solvent, and then by sterilizing the resulting mixture, for example, by filtration or irradiation. The methods of manufacture of sterile powders for the preparation of sterile injectable solutions are well known in the art and include vacuum drying and freeze-drying techniques. In general, the dosage of the particle will vary depending on considerations such as age, sex and weight of the patient, as well as the particular pathological condition suspected to affect the patient, the extent of the disease, or the area(s) of the body to be examined. Factors such as contra-indications, therapies, and other variables are also to be taken into account to adjust the dosage of the agent to be administered.

The invention will be further illustrated by the following figures and examples. However, these examples and figures should not be interpreted in any way as limiting the scope of the invention.

### FIGURES

FIG. 1. Crosslinking of the polysaccharides (dextran and fucoidan) with STMP in alkaline conditions.

FIG. 2. Evaluation of the SPs interactions with P-Selectin. A. Adhesion of the Control-SPs or Fuco-SPs on the coating of the recombinant P-Selectin in the microfluidic assay under arterial and venous flow conditions (n=4). B. Concentration dependent binding of the Fuco-SPs onto the coating of the P-Selectin at a range of concentrations. C. Fucoidan pre-injection inhibited Fuco-SPs adhesion onto the P-Selectin. D. Comparison of the Fuco-SPs binding to other selectins: E- and L-Selectin.

FIG. 3. A. In vitro rtPA release from rtPA-encapsulated Fuco-SPs by flow cytometry. B-C. Amidolytic activity measured by the PefaFluor® fluorogenic assay. B. The curves correspond to the fluorescence release and are correlated to the enzymatic velocity over 90 min. C. Corresponding quantitative analysis normalized to free rtPA at the same concentration at 90 min. D. Fibrinolytic activities of the SPs determined by a fibrin-plate agarose assay. The quantitative analysis normalized to free rtPA at the same concentration.

FIG. 4. Adhesion of the SPs over activated platelet aggregates. Visualization by fluorescent microscopy of the attached unloaded SPs was carried out on the microchannels after formation of the platelet aggregates. The figure displays corresponding quantification of integral density of the unloaded (n=6) and rtPA-loaded (n=4) Control-SPs and Fuco-SPs in ImageJ.

FIG. 5. Thrombolytic efficacy in vivo in the murine ischemic stroke model. A. Cerebral blood flow reperfusion monitored by the laser speckle contrast imaging during the 40-min treatment. B. Quantification of the volume of the infarct lesion 24 h post-stroke detected by MRI. C. Quantification of the BBB permeability at day 4 post-stroke detected by MRI.

FIG. 6. Cytocompatibility of polysaccharide submicronic particles from other polymers or their mixtures. Cytocompatibility of the different types of the SPs on HUVECs by Resazurin assay.

### EXAMPLE

### Methods

Materials: Dextran 40 kDa, TRITC-dextran 40 kDa and carboxymethyl dextran (CM) 40 kDa were provided by TdB Consultancy (Uppsala, Sweden). Pullulan 20 kDa was provided by Hayashibara (Okayama, Japan). Fucoidan (Mn=18 kDa/Mw=104 kDa) was a gift from Algues & Mer (Ouessant, France). Sodium trimetaphosphate (STMP), methylene blue hydrate, and Human Serum Albumin (HSA) were purchased from Sigma-Aldrich (Saint-Quentin-Fallavier, France). Polyglycerol polyricinoleate (PGPR) was obtained from Palsgaard France S.A.S. (Lyon, France). Vegetable (sunflower) oil was purchased from a local supermarket. The SPs were encapsulated with commercially available rtPA (Actilyse®, Boehringer Ingelheim) that was reconstituted at 1 mg/ml, aliquoted, and stored at −80° C. Chromatography paper was obtained from GE Healthcare (Chicago, Illinois, United States). Fibrillar type I collagen Horm® was obtained from Takeda (Linz, Austria). 96-Well Cell Culture Plates (Costar) were obtained from Corning Incorporated. PPACK (Phe-Pro-Arg-Chloromethylketone) 75 μM tubes were purchased from Cryopep (Montpellier, France). Flow chambers (Vena8 Fluoro+) were provided from Cellix Ltd (Dublin, Ireland).

Submicroparticle synthesis: Polysaccharide submicroparticles (SPs) were obtained via a water-in-oil (w/o) emulsification combined with a crosslinking process. Polysaccharide solution was prepared as a mixture of dextran 40 and 5% TRITC-dextran 40 (for fluorescent SPs) at 300 mg/ml, 6 M NaCl. To synthesize functionalized SPs with fucoidan (Fuco-SPs), 10% w/w of fucoidan was added.

First, the organic phase of 15 ml of sunflower oil and 6% w/v PGPR in Falcon® 50 ml was prepared and cooled down for 20 min at −20° C. In the meantime, 1,200 mg of the polysaccharide solution was incubated with 120 ml of 10 M NaOH under magnetic stirring for 10 min. 240 ml of STMP solution (30% w/v in water) was added into the aqueous phase under magnetic stirring and mixed for 20 seconds on ice. Next, emulsification was achieved by the dropwise injection of 600 ml of the aqueous phase into the organic phase and dispersed with a stand-disperser (Polytron PT 3100, dispersing aggregate PT-DA 07/2EC-B101, Kinematica, Luzernerstrasse, Switzerland) at 30,000 rpm for 4 min on ice. The obtained w/o emulsion was transferred into 50° C. for the crosslinking reaction of polysaccharides with STMP for 20 min. The crosslinked suspension was washed in 30 ml PBS 10×for 40 min under high magnetic stirring at 750 rpm. The mixture was then centrifuged (BR4i, JOUAN SA, Saint Herblain, France) for 10 min at 3,000 g in Falcon tubes. The organic phase was recovered and ultracentrifuged (Optima MAX-XP, Ultracentrifuge, Beckman Coulter, Brea, California, United States) for 45 min at 15,000 g. The obtained pellet was washed by ultracentrifugation 2 times in 0.04% Sodium Dodecyl Sulfate (SDS) solution and then 2 times in ultrapure water to purify the SPs. The resulting SPs were suspended in water or 0.9% NaCl with 0.02% Tween 20 (Sigma) and stored at 4° C.

TABLE 1
The synthesis parameters for the polysaccharide SPs.

| Synthesis | Polysaccharide | STMP, | NaOH | Phase ratio, | | | |
|---|---|---|---|---|---|---|---|
| method | [c], mg/ml | [c], mg/ml | [c], M | (Aq/ Org/) | Surfactant | Homogenization | Crosslinking |
| W/O | 300 | 56.11 | 1.15 | 4% w/v | PGPR, 6% | 30,000 rpm, | 20 min, 50° C. |
| emulsion / | | | | | | 4 min, 4° C. | |
| crosslinking | | | | | | | |

Abbreviations; W/O, water-in-oil; Aq: aqueous; Org: organic.

Cell culture and cytotoxicity assay: To evaluate the cytotoxicity of the SPs, Fluorometric Cell Viability Assay (Resazurin) was used on confluent Human Umbilical Vein Endothelial Cells (HUVEC). The cells were cultured in DMEM supplemented with 10% (v/v) fetal bovine serum, 4 mmol of 1-glutamine, 100 units/mL of penicillin, and 100 μg/mL of streptomycin and kept in an incubator at 37° C. in a humidified atmosphere of 5% CO₂. Cells were seeded into 96-well plates to adhere, 10,000 cells per well. Following 24 h of incubation to reach ˜80% confluency, the medium in the wells was changed to the one containing the SPs at concentrations ranging from 0.1 to 1.5 mg/ml and cultured for another 24h. Culture media were used as a positive control. Next, the medium was replaced with 100 μL 10% Resazurin solution, and the plates were covered in foil and incubated for 2h. The fluorescent signals of the Resazurin were monitored using 540 nm excitation and 590 nm emission wavelengths on Infinite® 200 PRO microplate reader (TECAN Group Ltd., Mannedorf, Switzerland). The obtained fluorescence (Fl) values were blank corrected; and the relative cell viability was expressed as Fl_{SPs}/Fl_{control}×100%, where Fl_{control} was obtained in the absence of the SPs. The experiment was performed in hexaplicate.

To examine the potential cell cytoskeleton organization mediated by Fuco-SPs, HUVEC cells were cultured in 8-well Lab-Tek II Chamber Slide w/Cover (Lab-Tek®, Thermo Fischer Scientific, Massachusetts, United States) with 10,000 cells per well. The wells' medium was changed 24 h after to the one containing TRITC-Fuco-SPs at 1.5 mg/ml and was incubated for another 24 h. Cells cultured in the medium without the SPs were set as control. Next, cells were fixed with 4% paraformaldehyde for 30 min at room temperature (RT). After rinsing with PBS, cells were labeled and permeabilized with the 200 μl mixture of FITC-Phalloidin (1:200, Sigma-Aldrich, USA)/DAPI (1:100, Thermo Fisher Scientific, Massachusetts, United States)/0.01% v/v Tween 20 in PBS and incubated under low agitation for 1h at RT. The cells were afterward washed 3 times with PBS. The support of the chamber slides was removed, and the slides were mounted with a few drops of the aqueous mounting medium and kept at 4° C. until visualization with the confocal microscope (Zeiss LSM 780, lena, Germany).

Hemocompatibility test: Hemolysis assay was adapted from the publication and performed on washed isolated murine erythrocytes. Murine blood was collected in sodium citrate 3.8% (w/v) and centrifuged at 800 g for 5 min to isolate red blood cells. The supernatant was removed, and the pellet of erythrocytes was resuspended at 20% (v/v) in distilled water (positive control, 100% hemolysis), normal saline (negative control, no hemolysis), and the Fuco-SPs at the concentrations from 0.1 to 1.5 mg/ml in Eppendorf. The tubes were incubated on a rotator at 37° C. for 1.5 h and then centrifuged at 3,000 g for 5 min. The absorbance (A) of the supernatants was measured on Infinite® 200 PRO microplate reader (TECAN Group Ltd., Mannedorf, Switzerland) at 590 nm. Each sample was run in triplicate. The percentage of hemolysis was determined by the formula: Hemolysis degree (%)=100%×(Aₛₐₘₚₗₑ−A_{negative control})/(A_{positive control}−A_{negative control}).

Physico-chemical characterization: The submicroparticle (SP) formulations were studied for particle morphology, size and zeta potential distributions, mass concentration, and elemental composition.

Particle morphology was visualized by Transmission Electron Microscopy (TEM) (Philips FEI Tecnai 12, Amsterdam, Netherlands), negatively stained with 1% (w/v) uranyl acetate for 5 minutes, and Environmental Scanning Electron Microscopy (ESEM) (Philips XL30 ESEM-FEG, Amsterdam, Netherlands). Hydrodynamic size was measured by Dynamic Light Scattering (DLS) or by Laser Diffraction (Zetasizer Nano ZS or Mastersizer 3000, Malvern Instrument SARL, Orsay, France respectively). Zeta potential (ζ-potential) was measured by Electrophoretic Light Scattering (ELS) (Zetasizer Nano ZS, Malvern Instruments SARL, Orsay, France). Samples were dissolved in distilled water or saline for size and in 1 mM KCl for ζ-potential determination. All runs were performed at 25° C. in triplicate.

Mass concentration was determined by freeze-drying. An elemental analyzer-mass spectrophotometer was used for the quantification of the sulfur (presence of fucoidan). To prove the crosslinking with STMP, total reflection X-ray fluorescence spectroscopy (TXRF) technique was applied to quantify the phosphorus content on the SPs (S2 PICOFOX Bruker, Massachusetts, United States).

For Fourier transform infrared (FTIR) spectroscopy measurement, ThermoNicolet AVATAR 370 FTIR spectrometer (Thermo Electron Corporation, Waltham, MA) was used. The samples were blended with KBr and compressed to form a pellet. The transmission spectra were obtained from 400 to 4000 cm⁻¹ with a resolution accuracy of 4 cm⁻¹.

Sulfate and fucoidan quantification: The sulfate content of fucoidan was determined by a semi-quantitative solid-phase colorimetric assay [29]. Briefly, 5 μL of Fuco-SPs in suspension at a concentration of 2 mg/mL were dropped on a piece of Whatman Chromatography paper grade 1. It was repeated 5 times on the same point, allowing the paper to dry at 50° C. in between. The paper was first soaked into a methanol/acetone (6:4) solution for 3 min and then into a methanol/acetone/water (6:4:15) solution with 50 mM HCl and 0.1% w/w methylene blue for 10 min. Finally, the paper was extensively washed with acetic acid/methanol/acetone/water (5:6:4:75) until no coloration was detected in the washing solution. The paper was then transferred to the Eppendorf, containing 0.5 mL methanol with 2% w/v SDS, and incubated for 15 min at 50° C. 0.2 mL of the extracted dye was placed in a 96-well plate, and its concentration was determined by reading absorbance at 663 nm on an Infinite® 200 PRO microplate reader (TECAN Group Ltd., Mannedorf, Switzerland). Standard curves were obtained from fucoidan in solution with known concentrations.

Loading rtPA on the SPs: rtPA was immobilized onto the SPs by adsorption. 100 μl of SPs (5 mg/ml) was mixed with 100 μl of rtPA (1 mg/ml) in ultrapure water and then incubated for 1h at RT. Free unabsorbed rtPA was removed by 3 cycles of ultracentrifugation (15 min, 15,000 g). The SPs with adsorbed rtPA (rtPA-SPs) were resuspended in water and used for the drug loading efficiency quantification.

Drug encapsulation efficiency: The amount of rtPA loaded on the SPs was measured using the Pierce BCA protein assay kit (Life Technologies SAS, Courtaboeuf, France). Briefly, 200 μl of working reagent was added to 25 μl of each sample in 96 well-plate. The absorbance at 562 nm was read on the Infinite® 200 PRO microplate reader (TECAN Group Ltd., Mannedorf, Switzerland) after 30 min of incubation at 37° C. and cooling to RT for 10 min. The concentration of the drug was extrapolated by a calibration curve prepared with different concentrations of rtPA.

In vitro rtPA release: The release of rtPA from the Fuco-SPs was assessed by flow cytometry [30]. FITC-rtPA (Abcam, Cambridge, United Kingdom) at 1 mg/mL was placed in contact with TRITC Fuco-SPs at 5 mg/mL for 1h at RT. The suspensions were added to tubes pre-filled with 400 mL of saline and placed under gentle agitation at 37° C. At each time point of 0, 15, 30, 45, 60, and 90 min, the tubes were analyzed with a BD FACS Aria™ III flow cytometer (Becton Dickinson, New Jersey, United States). The TRITC-Dextran, excited by a 543 nm laser, was detected at 569 nm while the FITC-rtPA, excited at 480 nm, was detected on a 530/30 nm PMT. Flow cytometry analyses were performed in triplicates with Diva software (Becton Dickinson). The protein release curve was obtained by normalizing the values of Mean Fluorescence Intensity (MFI) of the FITC-rtPA still associated with TRITC-Fuco-SPs.

In vitro amidolytic activity of rtPA-loaded SPs: Amidolytic activity of rtPA loaded SPs was assessed with the fluorogenic substrate PefaFluor® tPA (Cryopep, Montpellier, France). 2.5 μL of samples at 20 μg/ml was put in contact with 97.5 L of 100 mM HEPES buffer (pH 8.0, 154 mM NaCl, 0.1% HAS) in the 96-well plate. After the addition of 10 μL PefaFluor® at 1 mM, a kinetic profile was obtained by measuring the fluorescence level at 440 nm every 2 min for 90 min at 37° C. with Infinite® 200 PRO microplate reader (TECAN Group Ltd., Mannedorf, Switzerland). Free rtPA was used at the same concentration based on the Pierce BCA protein assay. Increase of fluorescence corresponded to the fluorogenic peptide substrate hydrolysis by rtPA. Enzymatic activity was determined from the resulting kinetic profile and compared to the one of free rtPA.

In vitro fibrinolytic activity of rtPA-loaded SPs: To assess the fibrinolytic activity of rtPA-loaded SPs, a fibrin lysis clot experiment was performed. 5 ml of TRIS Buffer with 3% w/v low melting agarose (Carl Roth GmbH p Co. KG, Karlsruhe, Germany) were heated to 65° C. 5 ml of Fibrinogen (from human plasma, Sigma Aldrich) solution in TRIS buffer at 5 mg/ml was slowly heated to 37° C. Once the agarose solution reached 65° C., it was cooled to 37° C., and 2.5 U of thrombin (from human plasma, Sigma Aldrich) was added. Next, a fibrinogen solution was slowly added into the agarose/thrombin mixture under gentle agitation to avoid the formation of bubbles. The reaction mixture was poured into a 9 cm Petri dish and cooled at 4° C. for 30 min until the fibrin clot became visible. On the solidified agarose gel, round wells were formed using a 3 mm punch as sample reservoirs. 5 μl of each sample at 45 μg/ml was dropped into the wells and incubated overnight at 37° C. in a humid environment. The degree of fibrin lysis was quantified with ImageJ by comparing the size of the fibrinolysis circle of the samples and free rtPA at the equivalent concentration based on the Pierce BCA protein assay.

Flow microchamber experiments: An in vitro flow adhesion assay was performed to evaluate the affinity of the Fuco-SPs with their molecular target. Micro-channels of Vena8 Fluoro+chambers (width: 0.04 cm, height: 0.01 cm, and length: 2.8 cm; Cellix Ltd, Dublin, Ireland) were coated overnight with recombinant human P-selectin, L-selectin or E-selectin (R&D systems France, Lille, France) at 100 μg/mL and left overnight at 4° C. To confirm the concentration-dependent binding of Fuco-SPs to P-selectin, some channels were coated with P-selectin at a range of concentrations (5, 25, 50, and 100 μg/mL). Channels were then washed with NaCl 0.9% and further incubated with HSA at 10 μg/mL for 2h.

A suspension of fluorescently labeled Control-SPs or Fuco-SPs at 1 mg/ml in saline was passed through the channels for 5 min at arterial and venous flow conditions (shear stress 67.5 dyne/cm² and 6.75 dyne/cm²) using an ExiGo™ pump (Cellix Ltd, Dublin, Ireland). For the competitive binding experiment, fucoidan solution (10 mg/ml) was injected 5 min prior to the Fuco-SPs at the same rate. Then, all the channels were washed with NaCl 0.9% for 1 min. The binding of the adhered SPs was visualized in real-time under fluorescence microscopy (Axio Observer, Carl Zeiss Microimaging GmbH, lena, Germany). For the quantitative analysis, the number of fluorescent SP clusters on each channel was measured using the “Analyze particles” tool in the image analysis software ImageJ (NIH, Bethesda, U.S.) with a 4-pixel threshold to eliminate the background noise.

To further investigate the binding efficiency of unloaded and loaded SPs to activated platelets, the microchannels of Vena8 Fluoro⁺ were coated with 50 μg/mL of fibrillar type I collagen Horm® overnight at 4° C. and rinsed with NaCl 0.9% before use. Human whole blood (EFS, Bichat Hospital, Paris, France), collected in the PPACK tubes and labeled with 5 mM DIOC6 (Life Technologies SAS, Saint-Aubin, France), was perfused at arterial shear stress for 5 min to induce platelet activation and aggregation. Platelet aggregation through contact with collagen was visualized in real-time with phase-contrast microscopy (Axio Observer, Carl Zeiss Microscopy, Oberkochen, Germany). After rinsing with NaCl 0.9%, fluorescent Control-SPs or Fuco-SPs (unloaded or loaded with rtPA) at 1 mg/ml were injected into the channels in saline for 5 min. Their accumulation onto activated aggregates was monitored in real-time. Channels were then washed for 1 min with NaCl 0.9%. Finally, the MFI of the fluorescent SPs that are bound to the platelets on each channel was analyzed with ImageJ. Intensity settings were kept the same for both types of SPs.

Animals and thrombin stroke model in vivo: Animal experiments were carried out on male Swiss wild-type mice (15-18 weeks old; 35-45 g; CURB, Caen, France). All experiments were performed following the French (Decree 87/848) and the European Communities Council (2010/63/EU) guidelines and were approved by the institutional review board (French ministry of Research). All the experiments were validated by Normandy's local ethical committee (CENOMEXA) registered under the reference number APAFIS #13172. Anesthesia was induced by the application of 5% isoflurane (Aerrane, Baxter) and maintained by 2% isoflurane in a mixture of O₂/N₂O (30%/70%).

Mice were placed in a stereotaxic device, then a small craniotomy was performed, the dura was excised, and the middle cerebral artery (MCA) was exposed. To induce the MCA occlusion, the coagulation cascade was triggered by the pneumatical injection of 1 μL murine a-thrombin (1 IU; Stago BNL) with a glass micropipette, as previously described [31]. Successful MCA occlusion was confirmed by the Laser Doppler flowmeter (Oxford Optronix). For the treatment, the animals were intravenously injected through a tail vein catheter (200 μL, 10% bolus, 90% infusion over 40 minutes) with either saline (n=5) or rtPA-Fuco-SPs (Actilyse® rtPA at 10 mg/kg) (n=6) 20 minutes after thrombus formation. Brain perfusion was monitored by Laser Speckle Contrast Imager (MOOR FLPI-2, Moor Instruments) throughout the treatment. Region of interest (ROI) was selected on the ipsilateral to occlusion and contralateral hemispheres to monitor the relative cerebral blood flow in the affected region (Flₜ=100%×Flᵢₚₛᵢ/Fl_{contra}). The post-stroke reperfusion was expressed as a Growth Rate (GR) of the blood flow increase in the ipsilateral ROI to contralateral one at a time point, and it was quantified as GR (%)=100%×(Flₜ₂−Flₜ₁)/Flₜ₁.

Magnetic resonance imaging acquisition and analysis: Mice were anesthetized with 5% isoflurane and maintained with 1.5-2% isoflurane in a mixture of O₂/N₂O (30%/70%) during the acquisitions. Experiments were carried out on a Pharmascan 7T (Bruker Biospin, Wissembourg, France). Three-dimensional T2-weighted images were acquired using a Multi-Slice Multi-Echo sequence (TE/TR 33 ms/2.500 ms) 24 h after the stroke. Lesion volumes were quantified on these images using ImageJ software (slice thickness 0.5 mm). Magnetic resonance angiography was performed using a 2D-TOF sequence (TE/TR 12 ms/7 ms) 24 h after ischemia, and the recanalization status of the MCA was determined blindly from the analysis of the merged MCA angiograms with maximum intensity. The angiographic score is based on the TICI (Thrombolysis in Cerebral Infarction) grade flow scoring (from Score 0: no perfusion to Score 3: full recanalization). For the in vivo detection of the BBB permeability, three dimensional T1 FLASH sequences (spatial resolution 70 mmx 70 mm; TE/TR 4.46/15; 3 averages; 4 min 2 s) were used, 15 min after the intravenous injection of 200 μl of a solution containing 50 μl of gadolinium chelate (DOTAREM) diluted in saline. BBB leakage was measured 4 days after the stroke induction, and its volume was quantified using ImageJ.

Thrombus targeting by Fuco-SPs in a murine model of venous thrombosis: Animal studies were done following principles of laboratory about animal care and with the approval of the animal care and use committee of the Claude Bernard Institute (APAFIS #8724, Paris, France). FeCl₃-induced in vivo thrombosis model on mesenteric vein was carried out on C57BL/6 male mice (EJ, Le Genest, St-Berthevin, France) aged 5-8 weeks. Mice were anesthetized with an intraperitoneal injection of ketamine (100 mg/kg) and xylazine (10 mg/kg). After midline abdominal incision, the mesentery was exposed, and vessels were visualized by an intravital microscope (Leica MacroFluo, Leica Microsystems SAS, Nanterre Cedex, France) using Orca Flash 4.0 scientific CMOS camera (Hamamatsu Photonics France SARL, Massy, France). For green fluorescent labeling of mitochondria of platelets and leucocytes, DIOC6 (Life Technologies SAS, Saint-Aubin, France) at 25 mM was retro-orbitally injected. The mesentery vein was covered with a 1 mm large Whatman chromatography paper that was prior soaked in a 10% w/v iron chloride (Sigma-Aldrich) solution for 1 min and then washed with saline. The formation of non-occlusive thrombi was monitored in real-time by fluorescence microscopy by the accumulation of fluorescently labeled platelets. TRITC fluorescent-labeled Control-SPs or Fuco-SPs were retro-orbitally injected 10 min after thrombus initiation with the volume of 150 μL (5 mice per group).

For histological evaluation, mice were sacrificed with pentobarbital overdose 5 min after administration of SPs. The affected part of the mesenteric vein was cut, washed in 0.9% NaCl, fixed in paraformaldehyde 4% (w/v), and frozen. The vein samples were cryosectioned at 10 μm thickness. The cell nuclei of a venous vascular wall were labeled with DAPI (Thermo Fisher Scientific, Massachusetts, United States) contained in a mounting medium (Vecto laboratories). The samples were observed by fluorescence microscopy. For the quantitative analysis, normalized MFI of the TRITC signal from SPs was expressed, defined as total TRITC fluorescence intensity divided by the size of the thrombus area on 2 slides from each mouse with the ImageJ (NIH, Bethesda, U.S.).

Statistical analysis: Quantitative data were expressed as mean±standard error of the mean (SEM) (n≥3). Statistical tests were carried out with GraphPad Prism 8 (GraphPad Software, Inc., La Jolla, U.S.) with a 95% confidence level. Kolmogorov-Smirnov normality test was utilized to examine if variables are normally distributed. Normally distributed data were analyzed then with unpaired t-test or one-way analysis of variance (ANOVA) with post hoc Turkey's test. The Mann-Whitney U test was applied otherwise. The p-values of * p<0.05; ** p<0.01; *** p<0.001 were considered statistically significant.

### Results and Discussion:

### Submicroparticle Synthesis and Characterization

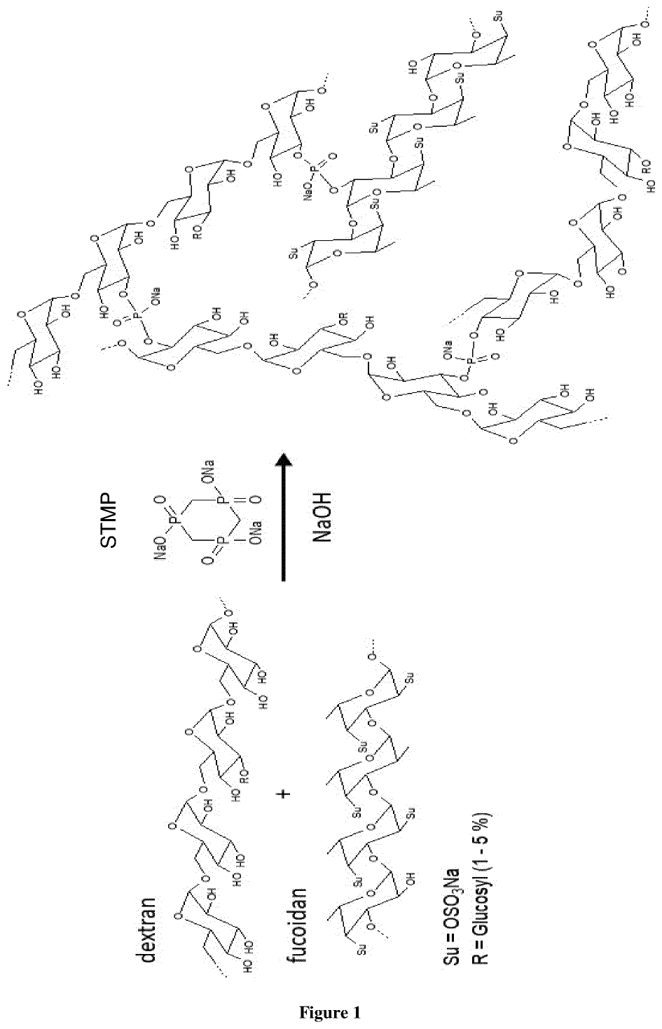

Novel polysaccharide SPs were elaborated by a simple and reproducible two-step synthesis process influenced by [16,17]. First, a stable w/o miniemulsion of the aqueous phase with hydrophilic polysaccharides and vegetable (sunflower) oil was prepared. Chemical crosslinking of polysaccharides with the crosslinking agent STMP under alkaline conditions (FIG. 1) produced a suspension of uniform SPs. We refer to the hydrogel particles only from dextran as Control-SPs, and from a mixture of dextran and fucoidan as Fuco-SPs in this EXAMPLE. To ensure the in vivo safety during the preparation of the drug delivery platform, low molecular weight dextran 40 kDa of clinical-grade was utilized without any chemical modification. Having a large number of hydroxyl groups, dextran is a suitable compound for subsequent chemical crosslinking with STMP [25], an FDA-approved non-toxic food additive [32]. Fucoidan, a murine sulfated polysaccharide, is approved as a pharmaceutical compound and exhibits a nanomolar affinity to P-selectin [34], thus, it served as a targeting ligand to thrombi. A recently completed phase 1 clinical trial of fucoidan radiolabeled by Technetium-99m reported its safety and favorable biodistribution as a potential diagnostic agent for the imaging of thrombosis [35]. Hence, both natural polysaccharides applied in this study are affordable, biodegradable, biocompatible compounds, non-immunogenic, and approved for clinical applications. Intriguingly, several articles claim that dextran and fucoidan [37,38] themselves exert some antithrombotic action that makes them an excellent starting material for the carrier.

Instead of commonly used organic solvents, sunflower oil was utilized as an emulsion continuous phase. The choice of the stabilizing agent plays an important role in reducing the interfacial tension and Laplace pressure when fabricating a stable emulsion and future nanocarrier. In this work, we selected a potent oil-soluble nonionic surfactant for stabilizing w/o emulsions—PGPR, which is also recognized by the FDA as a safe compound and is frequently used as an emulsifier in the food production industry [39]. In addition, to counteract the Ostwald ripening of water droplets, 6 M NaCl was added to the aqueous phase as an osmotic agent to adjust the osmotic gradient and to stabilize the w/o emulsion further. Overall, multiple parameters were optimized to obtain a stable and homogenous miniemulsion and subsequent nano-delivery system. It was found that the size of the templating droplet and the ultimate hydrogel SPs being directly proportional to the polysaccharide molecular weight and inversely proportional to the amount of surfactant and crosslinking agent as well as homogenization speed (data not shown).

The polysaccharide nature of the particles was proven by comparing their FTIR spectra with the equivalent ones of the polysaccharide themselves with a strong peak, corresponding to the —OH bond (data not shown).

ESEM and TEM images revealed a well-defined spherical morphology and uniform size distribution of SPs (data not shown). Functionalized Fuco-SPs contained 8.60±0.01% of fucoidan in a mass of the total SPs weight, determined by elemental analysis of sulfur, and 9.30±1.07% of fucoidan by quantification of the sulfate content by a semi-quantitative colorimetric assay. In such a way, two different techniques estimated ˜9% fucoidan composition in the SPs. The SPs exhibited the hydrodynamic size of 674.87±59.35 nm (Control-SPs) and 708.48±40.00 nm (Fuco-SPs) determined by DLS. It is essential to highlight that a relatively large size of the SPs might limit the accumulation of associated rtPA within the brain parenchyma and reduce the risk of hemorrhagic events. The negative ζ-potential of the SPs was −24.83±0.09 mV for Control-SPs and −27.07±0.39 mV for Fuco-SPs ensured colloidal stability as a result of the anionic nature of the fucoidan and the formation of the anionic phosphate functional groups, produced during the crosslinking reaction with STMP.

The obtained SPs preserved their integrity in a physiological solution of 0.9% NaCl. As hydrogel-based particles, they were able to swell in an aqueous medium while maintaining their network structure (data not shown). These soft particles resemble the networks of natural extracellular matrices that could minimize tissue irritation or cell adherence [40]. Size and zeta potential of both SPs remained to be stable at least 30 days at 4° C. storage (data not shown). In addition, adequate storage of the SPs can be ensured by freeze-drying with 5% (w/v) sucrose as a cryoprotectant and subsequent resuspension in an aqueous medium. The overall yield of the synthesis was 13.4±0.7 mg of SPs (data not shown).

It is important to remark that the submicronic particles could also be synthesized from other natural polysaccharides, such as pullulan, a mixture of dextran and pullulan, and a mixture of carboxymethyl dextran with dextran, via the miniemulsion/crosslinking protocol. This flexibility permits, for example, to modulate the mechanical properties of the obtained particles and introduce functional such as COOH onto the surface of the SPs that might be relevant for other applications. Table 2 summarizes experimental conditions used for the particle production in a reproducible way, with sizes in the submicronic range. Due to the anionic charges brought by STMP, all the SPs exhibited negative zeta potential.

TABLE 2
Formulation of polysaccharide submicronic particles

| | | | | | | | Zeta |
|---|---|---|---|---|---|---|---|
| | Organic | | | | | | potential, |
| Aqueous Phase | phase | V/N | D(0.1) | D(0.5) | D(0.9) | Span | mV |
| Pullulan 20 kDA | 6% PGPR | V | 0.708 | 1.01 | 1.47 | 0.753 | −29.8 |
| Fucoidan MMW | 15 mL SO | N | 0.669 | 0.810 | 1.16 | 0.669 | |
| (450 mg/ml, 6 M NaCl) | | | | | | | |
| 4.6% w/v STMP, 0.76 M NaOH | | | | | | | |
| Pullulan 20/Dextran 40 kDA | 6% PGPR | V | 0.877 | 1.22 | 1.77 | 0.885 | −29.6 |
| 65/35 | 15 mL SO | N | 0.776 | 1.03 | 1.42 | 0.883 | |
| Fucoidan MMW | | | | | | | |
| (450 mg/ml, 6 M NaCl) | | | | | | | |
| 4.6% w/v STMP, 0.76 M NaOH | | | | | | | |
| CM Dextran 40/Dextran 40 kDA | 6% PGPR | V | 0.645 | 0.898 | 1.25 | 0.669 | −29.9 |
| 20/80 | 15 mL SO | N | 0.557 | 0.735 | 1.03 | 0.642 | |
| Fucoidan MMW | | | | | | | |
| (300 mg/ml, 6 M NaCl) | | | | | | | |
| 4.6% w/v STMP, 0.76 M NaOH | | | | | | | |

Size and zeta potential of the SPs by laser diffraction and ELS, respectively.
D(0.1), D(0.5) and D(0.9) are the particle diameters at 10%, 50% and 90% of the particle size distribution by number (N) or volume (V).
Span refers to the width of the distribution.

### Biocompatibility of the SPs

The injectable hydrogel SPs were produced according to the green chemistry principles through the formulation method without the use of hazardous substances and organic solvents and were expected to be biocompatible.

An initial in vitro evaluation of biocompatibility of the developed SPs examined cyto- and hemocompatibility. The cytocompatibility of the SPs was assessed with a resazurin cell viability assay. Following 24 h exposure, Control-SPs and Fuco-SPs did not affect cellular viability and metabolic activity of HUVECs at concentrations ranging from 0.1 to 1.5 mg/ml, exhibiting an excellent cytocompatible profile (cell survival>90%, up to the highest tested concentrations of SPs) (data not shown). The upper limit for the tested concentration 1.5 mg/ml of the SPs was selected to surpass the tested concentrations for the majority of the nanosystems in vitro (typically, maximum 400 μg/ml) and the concentration of the SPs employed for further in vivo experiments in this work (71 mg SPs per 1 kg body weight or 1.1 mg SPs per 1 ml of blood). There was no significant difference between the Control-SPs and Fuco-SPs, as both of them did not provoke cytotoxicity. Additionally, high cell viability was similarly observed in the SPs from other polysaccharides (FIG. 6).

Since the SPs in this study were designed for intravenous administration and were expected to have direct contact with blood, the Fuco-SPs were examined for their blood-compatible behavior by a hemolysis test on isolated murine red blood cells in vitro (data not shown). Even at the highest concentration of 1.5 mg/ml, the SPs presented a hemolytic index 1.51±0.02%, which is below 2% and considered to be nonhemolytic according to ISO 10993-4 standard [42,43].

Morphology of the cells, co-cultured with Fuco-SPs, was visualized with confocal microscopy. No apparent morphological differences were revealed for HUVECs with Fuco-SPs and negative control (data not shown). FITC-Phalloidin staining was used to visualize a cytoplasm and DAPI for nuclei. Moreover, the SPs were internalized by endocytosis as the merged images of all three probes revealed colocalization of the particles within the dye in the cytoplasm. Collectively, these results suggest that the polysaccharide SPs have favorable biocompatibility for their application in vivo.

### Binding of SPs to P-Selectin In Vitro

Knowing that fucoidan was homogeneously distributed in the structure of the hydrogel Fuco-SPs and constituted ˜9% w/w of the composition, we investigated whether its quantity on the surface was sufficient for specific adhesion to its molecular target. While most of the publications assess targeting strategy in vitro in static conditions by flow cytometry or confocal microscopy [17,44,45], our group developed a robust and tunable dynamic microfluidic method to study the targeting efficacy for recombinant P-selectin or/and human activated platelet aggregates expressing P-selectin and previously validated it with fucoidan-coated nano-/microcarriers [18,19,28] (data not shown).

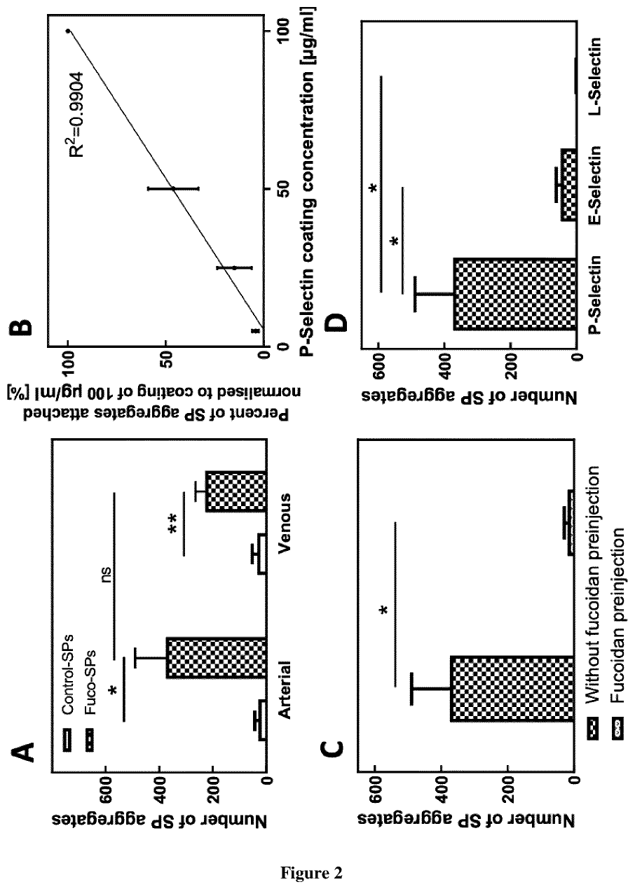

First, fluorescent Fuco-SPs and Control-SPs were injected in the microchannels coated with recombinant P-selectin under arterial or venous shear rates (67.5 dyne/cm² vs. 6.75 dyne/cm²), and their adhesion was visualized and quantified in real-time under fluorescence microscopy. According to obtained results, fluorescent Fuco-SPs depicted a significantly higher adhesion to P-selectin coating than Control-SPs both in arterial (374.25±115.33 adhered Fuco-SPs vs. 30.25±13.84 adhered Control-SPs, * p<0.05) and venous (228.25±36.67 adhered Fuco-SPs vs. 34.50±18.16 adhered Control-SPs, ** p<0.01) flow conditions (FIG. 2A). There was no significant difference between the fluorescent signal of Fuco-SPs accumulation for arterial and venous flow conditions. Fuco-SPs accumulation after injection onto P-selectin coating was in a linear dose-dependent manner as regards to the P-selectin concentration, R²=0.9904 (FIG. 2B). An experiment of competitive interaction illustrates that fucoidan solution pre-injection at 10 mg/ml considerably reduced the attachment of the Fuco-SPs onto the microchannels with P-selectin (374.25±115.33 vs. 19.75±10.06, * p<0.05) (FIG. 2C).

To establish the specificity of the Fuco-SPs binding to P-selectin, the targeting assay was extended to other members of the selectin family: E- and L-selectin [46]. The percentage of the Fuco-SPs adhered to the E- and L-selectin was normalized over the mean number of the attached Fuco-SPs to a P-selectin coating at the equivalent concentration. Indeed, only 12.73±3.66% of the SPs adhered to E-selectin and 0.26±0.19% to L-selectin coating (FIG. 2D). Thus, our results indicate that Fuco-SPs bind specifically to P-selectin but not to E- and L-selectins, and these results are in accordance with a previous work of our group published by Bo L. et al. of fucoidan-functionalized polymer microcapsules [18].

Overall, these findings are encouraging evidence of the sensitivity and selectivity of the Fuco-SPs, confirming fucoidan potential as a natural ligand of P-selectin.

rtPA Loading onto the SPs and its Release in Saline. In Vitro Thrombolytic Activity of Rt-PA-Loaded SPs

Due to rtPA low bioavailability and requirement of high dose administration, coupling this enzyme to the biocompatible carrier could overcome the drawbacks associated with a drug high dosage. Herein, an efficient rtPA encapsulation was achieved through the physical adsorption method due to electrostatic interaction [47]: the protein was put in contact with negatively charged polysaccharide SPs in the water at pH below rtPA isoelectric point IP=7.7 when rtPA presented a positive charge. Since adsorption is a mild drug encapsulation method, it can prevent rtPA from the disadvantages of the covalent bioconjugation such as changes in the protein structure and function that might result in its partial denaturation and loss of activity [49]. The nanogel nature of the SPs allowed reaching a high encapsulation efficiency of the rtPA of 64.78±2.16% and 81.04±1.86% for Control-SPs and Fuco-SPs, respectively. The confocal microscopy images of FITC-rtPA loaded onto TRITC-labelled Fuco-SPs revealed the uniform distribution of the rtPA within a porous structure of the hydrogel SPs, as evidenced by a green fluorescence from FITC-rtPA colocalized with the red fluorescence from the particles (data not shown).

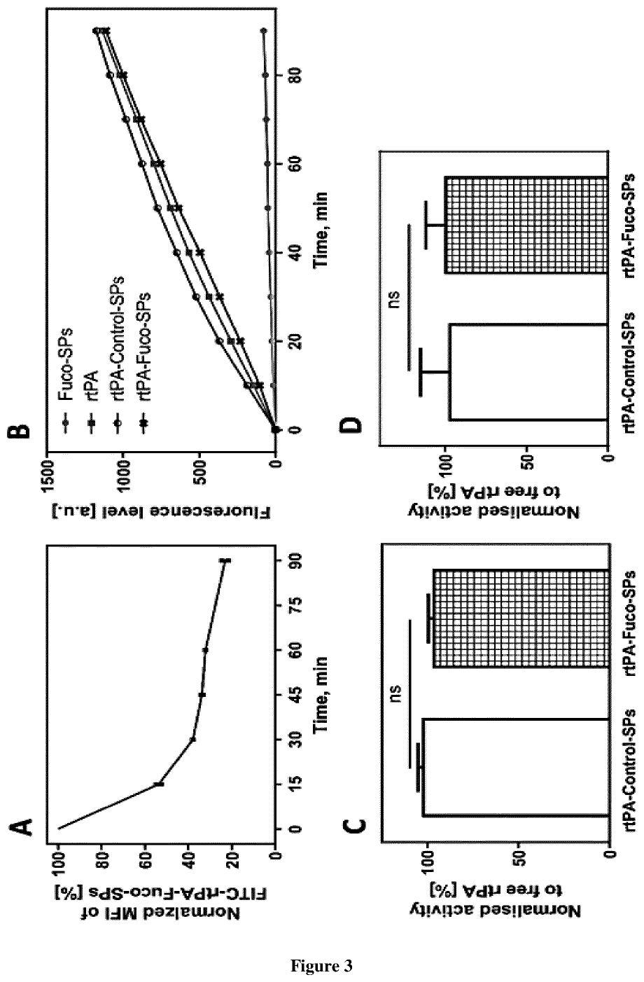

The release kinetics of rtPA from fucoidan-functionalized SPs was analyzed in vitro by flow cytometry in saline at 37° C. under gentle agitation by quantification of the MFI of the FITC-labelled rtPA associated with TRITC-fluorescent Fuco-SPs. FIG. 3A indicated a gradual and continuous sustained release of the lytic agent from the SPs during the observation period: 46.41±1.34% of the encapsulated protein was released during the first 15 min and 76.98±1.74% after 90 min. This release profile is classical for the hydrogels [50].

The thrombolytic activity of the rtPA-loaded SPs in vitro was analyzed as a combination of amidolytic and fibrinolytic activities and was reported in FIG. 3 (B, C, D). Amidolytic or enzymatic activity featured the ability of the proteolytic enzyme to hydrolyze the rtPA substrate. Interestingly, the amidolytic activities of rtPA on Control-SPs and Fuco-SPs were comparable to that of free rtPA (FIG. 3B & 3C). The fibrinolytic experiment in vitro of the rtPA-loaded SPs was performed in a fibrin plate assay. The results indicated full retention of fibrinolytic activity (FIG. 3D). Thus, rtPA loaded onto the SPs appeared to be able to diffuse into the fibrin-agarose matrix and to induce fibrinolysis in contact with fibrin. Comparing covalent vs. non-covalent conjugation approaches, Friedrich et al. documented that the adsorptive bound rtPA liberated faster from the particles and diffused more readily into the fibrin matrix than covalently bound rtPA which may also be beneficial in targeted thrombolysis. No significant difference was detected between both types of SPs enabling to utilize them in the following set of experiments.

Overall, the rtPA association with the SPs did not affect the drug amidolytic activity and fibrinolytic potential in our design. This result is in accordance with the most studies on nanogels suggesting that drug encapsulation via a passive diffusion into the preformed nanogels does not affect the secondary structure of the adsorbed protein and its biological activity [52].

Unloaded and rtPA-Loaded Fuco-SPs Adhere to Activated Platelet Aggregates In Vitro Under Arterial Flow

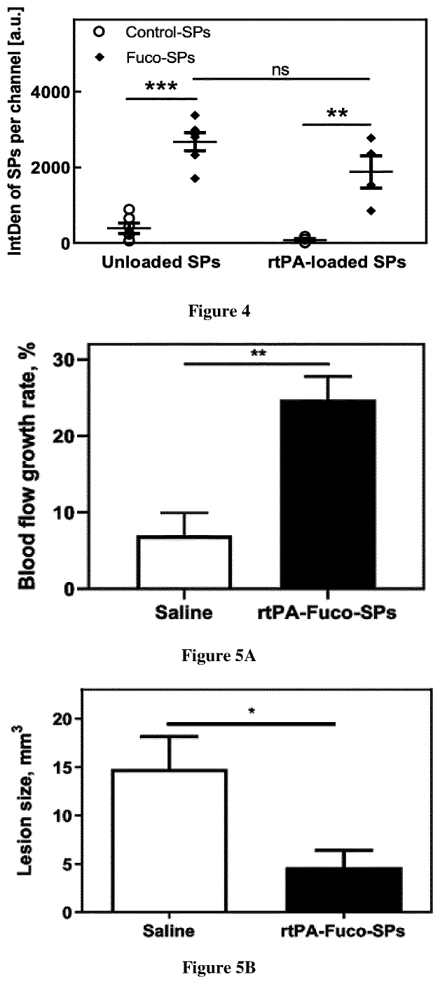

Because aggregation of activated platelets and platelet-mediated coagulation pathways are hallmark events in thrombosis, activated platelets are a suitable cellular target for carrier binding to thrombi [53]. Before the in vivo tests, we complemented the targeting evaluation with the second set of microfluidic experiments to validate Fuco-SPs capability to actively anchor onto the surface of activated platelets, expressing P-selectin. Thus, human whole blood was passed into collagen-coated microchannels to induce platelet activation and aggregation. Fuco-SPs or Control-SPs were then perfused at arterial shear stress (67.5 dyne/cm²), and the accumulation of the fluorescence from the adhered SPs was detected on the surface of activated platelet aggregates. By a quantitative analysis of the MFI (FIG. 4), it was revealed that Fuco-SPs adhered significantly more onto activated platelets than Control-SPs (2678.34±237.40 for Fuco-SPs vs. 392.44±137.15 for Control-SPs. *** p<0.001). Notably, adsorption of rtPA did not impair the Fuco-SPs clot-binding ability (1880.80±429.37 for rtPA-Fuco-SPs vs. 77.56±40.25 for rtPA-Control-SPs, ** p<0.01). There was also no significant difference between unloaded and rtPA-loaded Fuco-SPs adhering to the activated platelets.

To conclude, these in vitro experiments provided crucial evidence of molecular interaction and high affinity between the P-selectin on the activated platelets and fucoidan-functionalized SPs, which is maintained after loading of the thrombolytic agent. This finding presumes that the administration of the rtPA-Fuco-SPs could enable a specific delivery of the rtPA-immobilized SPs to the platelet-rich thrombus with higher drug accumulation.

### In Vivo Thrombolytic Efficacy

Whereas demonstrating the in vitro activity of the rtPA immobilized on the drug delivery system is important, the in vivo therapeutic effect is paramount. A murine thromboembolic stroke model was established by in situ injection of 1 IU of thrombin into the MCA by provoking a coagulation cascade and formation of a fibrin-rich clot in the lumen of the artery [54,55]. The treatment options—control saline or 10 mg/kg rtPA-Fuco-SPs—were intravenously injected 20 min after ischemic onset in accordance with rtPA clinical mode of administration: 10% bolus followed by 90% infusion. It is important to note that 10 mg/kg is a relevant dose in mice in place of 0.9 mg/kg in humans because of a lower sensitivity of human rtPA in murine plasma [56].

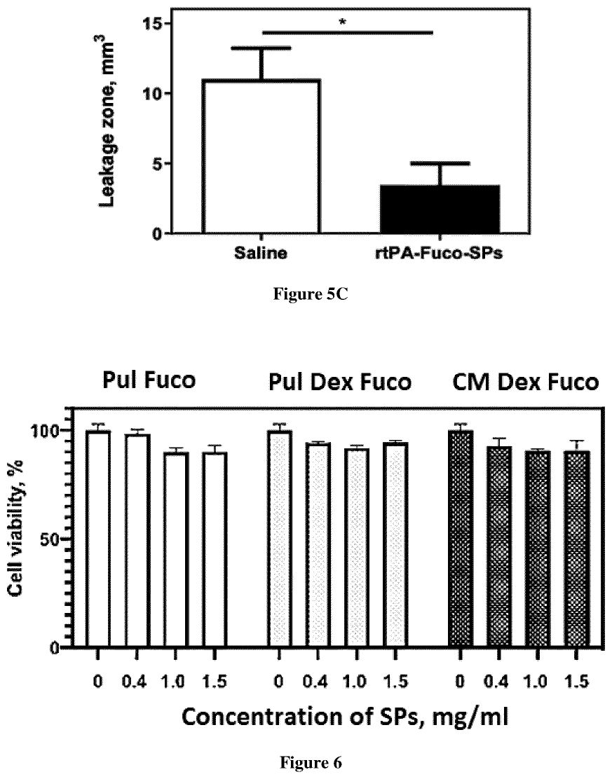

Cerebral blood flow was monitored throughout the treatment via laser speckle contrast imaging, a high resolution and high-speed technique that instantly visualizes microcirculatory tissue blood perfusion. The blood flow in the ipsilateral cerebral hemisphere was restored by 24.78±3.00% after 40 min treatment with rtPA-Fuco-SPs; by contrast, in the saline group the perfusion was improved only by 7.01±3.13% (FIG. 5A). The representative laser speckle multispectral imaging in the ipsilateral and the contralateral hemispheres are expressed at 0 min and 40 min (data not shown).

The prevailing method for assessment of the brain infarct volume is a brain tissue staining with 2% 2,3,5-triphenyltetrazolium chloride (TTC) which labels non-injured tissue and leaves the infarct area white. Several groups previously reported the reduction of the infarct zone in preclinical models by nanomedicine. For example, the magnetic iron oxide (Fc₃O₄)-microrods and polyacrylic acid-stabilized magnetic NPs conjugated with rtPA diminished the brain infarct lesion in FeCl₃ murine model of ischemic stroke of MCA. These designs, however, require an external magnet for targeting and to complement chemical lysis with rtPA with mechanical one of the magnetic rotation. Without any clinically approved medical device to impose a high magnetic force on the NPs in deep blood vessels, it would probably be desirable for nanocarrier formulation to avoid external assistance. Mei et al. stated that only the synergistic effect of rtPA-loaded polymer micelles and a reactive oxygen species (ROS)-eliminating antioxidant suppressed an infarct volume and improved neurological deficit after brain ischemia in the mouse model of photo-thrombotic MCA occlusion [59].

In our case, we utilized MRI imaging as a powerful technique to quantify the volume of brain damage. Using T2-weighted MRI sequences, we observed that the size of the ischemic brain lesions for the saline group was 14.80±3.34 mm³ whereas for the rtPA-loaded fucoidan-targeted SPs it was 4.63±1.59 mm^{3 24} h post-stroke (FIG. 5B). From the recent publication of the same group [55], it is well known that early intravenous administration of free rtPA at the dose of 10 mg/kg diminishes the lesion size by 26.2% in the thrombin model. Our data clearly stated that the rtPA-Fuco-SPs provided superior brain protection than a standard rtPA treatment by reducing the ischemic zone by 69%, almost 3-fold higher of free rtPA. Out of all treated animals with rtPA-Fuco-SPs, 66.6% of the cases had small lesion sizes (<3 mm³) and 33.3% had medium lesions (<11 mm³). In the saline treatment group, we recorded 60% severe (<20 mm³) and critical (>20 mm³) cases and no small lesions (data not shown). This therapeutic benefit could be ascribed to faster MCA reperfusion and, hence, prevention of the major brain injury due to higher rtPA accumulation on the thrombus site as to active targeting and specific P-selectin interactions of the Fuco-SPs.

To monitor the BBB integrity disrupted by ischemic stroke [60], gadolinium hypersignal was detected and quantified on T1-weighted MRI images at day 4 after stroke. Gadolinium extravasation from the blood into the brain parenchyma was unmistakably located at the ischemia-affected region where the BBB was compromised. rtPA-Fuco-SPs treatment group demonstrated a significant BBB preservation over saline with only a subtle BBB breakdown of 3.45±1.40 mm³ vs. 11.02±1.71 mm³ in a saline group (FIG. 5C). It is vital to underline that although in this article we utilized fucoidan on the SPs purely for the P-selectin targeting purpose, new avenues of research are deciphering its neuroprotective role, particularly after cerebral ischemic events as recently reported [61].

Similar to some untreated stroke patients, the blood clots were gradually lysed post-stroke in this murine model: at 24 h after thrombotic occlusion, 40% of mice exhibited a total (Score 3) recanalization and 60% partial perfusion (Score 1 and Score 2) of the MCA when injected with saline (data not shown). However, after the treatment with rtPA-Fuco-SPs, most of the cases were entirely recanalized (Score 3) with an absence of Score 1. These angiographic analyses were assessed by a blinded observer based on TICI grade flow scoring.

Overall, the apparent superiority of rtPA-Fuco-SPs to reduce the brain injury area in comparison with saline and rtPA at the same dose, combined with a favorable safety profile of the SPs. makes them a promising nanomedicine-based approach for the treatment of acute arterial thrombosis. We speculate that the submicron size of the particles as well as their active thrombus-targeting moiety should maintain rtPA-loaded carrier within the intravascular compartment to exert its thrombolytic activity. This should prevent the leakage of rtPA into the brain parenchyma, reducing the risks of NMDA receptors-mediated neurotoxicity and hemorrhages [62]. Further studies could compare a single or a double bolus route of administration of rtPA-Fuco-SPs due to the rtPA preservation by SPs, and, thus, a more comfortable treatment option for patients.

### Conclusions:

In the present study, we designed and fabricated fucoidan-functionalized 100% polysaccharide submicroparticles from biocompatible and FDA approved components as a P-selectin targeting drug delivery system for thrombolytic therapy. The physico-chemical properties and a biocompatibility analysis of these SPs were thoroughly evaluated, and a clinically used thrombolytic molecule—alteplase or rtPA—was effectively immobilized onto the SPs with full retention of its enzymatic and fibrinolytic potential in vitro and in vivo. The commercially available alteplase requires an excess of L-arginine (3.5 mg amino acid per 100 mg rtPA) to stabilize the formulation by enhancing its solubility and preventing aggregation. L-arginine, whose pK value (negative of the logarithm of the dissociation constant for the —COOH group) equals 2.17, bears at least two free primary amine groups and carries a positive charge at physiological pH. Classically, rtPA is loaded onto the carriers using the covalent bond formation via EDC/NHS reaction. Although Juenet et al. managed to adsorb rtPA onto the surface of the dextran-coated core-shell polymer NPs with a near-neutral ζ-potential, the presence of the free primary amines was required on chemically modified dextran. Comparing covalent vs. non-covalent methods to conjugate rtPA onto the polymer coating of magnetic NPs [63]. better loading efficiency was described with a covalent one vs. adsorption (98.6±0.8% vs. 47.7±5.4%), as well as superior amidolytic and fibrinolytic activities. In the present report, high encapsulation efficiency of alteplase (˜80%) onto the polysaccharide SPs was attained without any chemical modification of the dextran or fucoidan and despite a distinctively negative surface charge of the SPs, therefore, can be attributed to their hydrogel-based structure. The fibrinolytic drug was associated not only at the surface of the particles but it also diffused through the matrix of the porous SPs. fully preserving its thrombolytic potential.

We established in vitro by dynamic flow microchamber assays that the fucoidan-functionalized particles specifically adhered to the recombinant P-selectin in a dose-dependent manner, but not to E- and L-Selectins, and to human activated platelets. Finally, our findings revealed in the murine model of ischemic stroke that rtPA conjugation to the Fuco-SPs could enhance the thrombolytic activity of the clinical agent in vivo. The blood flow perfusion was restored more rapidly which resulted in smaller post-ischemic cerebral infarct lesions and higher BBB protection. In summary, we suggest that a hydrogel-based delivery system with fucoidan holds a significant promise to revolutionize the safety and efficacy of thrombolytic therapy. In the future research, our biocompatible Fuco-SPs could also efficiently vehicle other therapeutic or contrast agents in the vascular compartment to target P-selectin overexpressed pathologies, such as cardiovascular diseases or some cancers [65,66].

### REFERENCES

Throughout this application, various references describe the state of the art to which this invention pertains. The disclosures of these references are hereby incorporated by reference into the present disclosure.
[1] E. J. Benjamin, P. Muntner, A. Alonso, M. S. Bittencourt, C. W. Callaway, A. P. Carson, A. M. Chamberlain, A. R. Chang. S. Cheng, S. R. Das, F. N. Delling, L. Djousse, M. S. V. Elkind, J. F. Ferguson, M. Fornage, L. C. Jordan, S. S. Khan, B. M. Kissela, K. L. Knutson, T. W. Kwan, D. T. Lackland, T. T. Lewis, J. H. Lichtman, C. T. Longenecker, M. S. Loop, P. L. Lutsey, S. S. Martin, K. Matsushita, A. E. Moran, M. E. Mussolino, M. O'Flaherty, A. Pandey, A. M. Perak, W. D. Rosamond, G. A. Roth, U. K. A. Sampson, G. M. Satou, E. B. Schroeder, S. H. Shah, N. L. Spartano, A. Stokes, D. L. Tirschwell, C. W. Tsao, M. P. Turakhia, L. B. VanWagner, J. T. Wilkins, S. S. Wong. S. S. Virani, Heart Disease and Stroke Statistics-2019 Update: A Report From the American Heart Association, Circulation. (2019). https://doi.org/10.1161/CIR.0000000000000659.
[2] W. J. Powers, A. A. Rabinstein, T. Ackerson, O. M. Adeoye, N. C. Bambakidis, K. Becker, J. Biller, M. Brown, B. M. Demaerschalk, B. Hoh, E. C. Jauch, C. S. Kidwell, T. M. Leslie-Mazwi, B. Ovbiagele, P. A. Scott, K. N. Sheth, A. M. Southerland, D. V. Summers, D. L. Tirschwell, Guidelines for the Early Management of Patients With Acute Ischemic Stroke: 2019 Update to the 2018 Guidelines for the Early Management of Acute Ischemic Stroke: A Guideline for Healthcare Professionals From the American Heart Association/American Stroke Stroke. 50 (2019) c344-c418. https://doi.org/10.1161/STR.0000000000000211.
[3] J. Mican, M. Toul, D. Bednar, J. Damborsky, Structural Biology and Protein Engineering of Thrombolytics, Comput. Struct. Biotechnol. J. 17 (2019) 917-938. https://doi.org/10.1016/j.csbj.2019.06.023.
[4] R. Bhatia, M. D. Hill, N. Shobha, B. Menon, S. Bal, P. Kochar, T. Watson, M. Goyal, A. M. Demchuk, Low rates of acute recanalization with intravenous recombinant tissue plasminogen activator in ischemic stroke: Real-world experience and a call for action, Stroke. 41 (2010) 2254-2258. https://doi.org/10.1161/STROKEAHA.110.592535.
[5] A. M. Thiebaut, M. Gauberti, C. Ali, S. Martinez De Lizarrondo, D. Vivien, M. Yepes, B. D. Roussel, The role of plasminogen activators in stroke treatment: fibrinolysis and beyond, Lancet Neurol. 17 (2018) 1121-1132. https://doi.org/10.1016/S1474-4422(18)30323-5.
[6] A. Pal Khasa, Y. Pal Khasa, The evolution of recombinant thrombolytics: Current status and future directions, Bioengineered. 8 (2017) 331-358. https://doi.org/10.1080/21655979.2016.1229718.
[7] S. Liu, X. Feng. R. Jin, G. Li, Tissue plasminogen activator-based nanothrombolysis for ischemic stroke, Expert Opin Drug Deliv. 15 (2018) 173-184. https://doi.org/10.1080/17425247.2018.1384464.
[8] N. Korin, M. Kanapathipillai, B. D. Matthews, M. Crescente, A. Brill, T. Mammoto, K. Ghosh, S. Jurek, S. A. Bencherif, D. Bhatta, A. U. Coskun, C. L. Feldman, D. D. Wagner, D. E. Ingber, Shear-Activated Nanotherapeutics for Drug Targeting to Obstructed Blood Vessels, Science (80−). 337 (2012) 738-742. https://doi.org/10.1126/science.1217815.
[9] M. Colasuonno, A. L. Palange, R. Aid, M. Ferreira, H. Mollica, R. Palomba, M. Emdin, M. Del Sette, C. Chauvierre, D. Letourneur, P. Decuzzi, Erythrocyte-Inspired Discoidal Polymeric Nanoconstructs Carrying Tissue Plasminogen Activator for the Enhanced Lysis of Blood Clots, ACS Nano. 12 (2018) 12224-12237. https://doi.org/10.1021/acsnano.8b06021.
[10] J. Zhou, D. Guo, Y. Zhang. W. Wu, H. Ran, Z. Wang. Construction and evaluation of Fe₃O₄-based PLGA nanoparticles carrying rtPA used in the detection of thrombosis and in targeted thrombolysis, ACS Appl. Mater. Interfaces. 6 (2014) 5566-5576. https://doi.org/10.1021/am406008k.
[11] A. Friedman, S. Claypool, R. Liu, The Smart Targeting of Nanoparticles, Curr. Pharm. Des. 19 (2013) 6315-6329. https://doi.org/10.2174/13816128113199990375.
[12] W. jin Jeong, J. Bu, L. J. Kubiatowicz, S. S. Chen, Y. S. Kim, S. Hong. Peptide-nanoparticle conjugates: a next generation of diagnostic and therapeutic platforms?, Nano Converg. 5 (2018) 1-18. https://doi.org/10.1186/s40580-018-0170-1.
[13] L. Chollet, P. Saboural, C. Chauvierre, J.-N. Villemin, D. Letourneur, F. Chaubet, Fucoidans in Nanomedicine, Mar. 1-24. Drugs. 14 (2016) https://doi.org/10.3390/md14080145.
[14] L. Bachelet, I. Bertholon, D. Lavigne, R. Vassy, M. Jandrot-Perrus, F. Chaubet, D. Letourneur, Affinity of low molecular weight fucoidan for P-selectin triggers its binding to activated human platelets, Biochim. Biophys. Acta-Gen. Subj. 1790 (2009) 141-146. https://doi.org/10.1016/j.bbagen.2008.10.008.
[15] P. Saboural, F. Chaubet, F. Rouzet, F. Al-Shoukr, R. Ben Azzouna, N. Bouchemal, L. Picton, L. Louedec. M. Maire, L. Rolland, G. Potier, D. Le Guludec, D. Letourneur, C. Chauvierre, Purification of a low molecular weight fucoidan for SPECT molecular imaging of myocardial infarction, Mar. Drugs. 12 (2014) 4851-4867. https://doi.org/10.3390/md12094851.
[16] T. Bonnard, J.-M. Serfaty, C. Journe, B. Ho Tin Noc, D. Arnaud, L. Louedec, M. Derkaoui, D. Letourneur, C. Chauvierre, C. Le Visage, Leukocyte mimetic polysaccharide microparticles tracked in vivo on activated endothelium and in abdominal aortic aneurysm, Acta Biomater. 10 (2014) 3535-3545. https://doi.org/10.1016/j.actbio.2014.04.015.
[17] T. Bonnard, G. Yang, A. Petiet, V. Ollivier, O. Haddad, D. Arnaud, L. Louedec, L. Bachelet-Violette, S. M. Derkaoui, D. Letourneur, C. Chauvierre, C. Le Visage, Abdominal Aortic Aneurysms Targeted by Functionalized Polysaccharide Microparticles: a new Tool for SPECT Imaging, Theranostics. 4 (2014) 592-603. https://doi.org/10.7150/thno.7757.
[18] B. Li, M. Juenet, R. Aid-Launais, M. Maire, V. Ollivier, D. Letourneur, C. Chauvierre, Development of Polymer Microcapsules Functionalized with Fucoidan to Target P-Selectin Overexpressed in Cardiovascular Diseases, Adv. Healthc. Mater. 6 (2017) 1-11. https://doi.org/10.1002/adhm.201601200.
[19] B. Li, R. Aid-Launais, M.-N. Labour, A. Zenych, M. Juenet, C. Choqueux, V. Ollivier, O. Couture, D. Letourneur, C. Chauvierre, Functionalized polymer microbubbles as new molecular ultrasound contrast agent to target P-selectin in thrombus, Biomaterials. 194 (2019) 139-150. https://doi.org/10.1016/j.biomaterials.2018.12.023.
[20] I. Cicha, C. Chauvierre, I. Texier, C. Cabella, J. M. Metselaar, J. Szebeni, L. Dézsi, C. Alexiou, F. Rouzet, G. Storm, E. Stroes, D. Bruce, N. MacRitchie, P. Maffia, D. Letourneur, From design to the clinic: Practical guidelines for translating cardiovascular nanomedicine, Cardiovasc. Res. 114 (2018) 1714-1727. https://doi.org/10.1093/cvr/cvy219.
[21] K. Ganguly, K. Chaturvedi, U. A. More, M. N. Nadagouda, T. M. Aminabhavi, Polysaccharide-based micro/nanohydrogels for delivering macromolecular therapeutics, J. Control. Release. 193 (2014) 162-173. https://doi.org/10.1016/j.jconrel.2014.05.014.
[22] J. Zhang, W. Xia, P. Liu, Q. Cheng. T. Tahirou, W. Gu, B. Li, Chitosan modification and pharmaceutical/biomedical applications, Mar. Drugs. 8 (2010) 1962-1987. https://doi.org/10.3390/md8071962.
[23] H. J. Jin, H. Zhang, M. L. Sun, B. G. Zhang, J. W. Zhang, Urokinase-coated chitosan nanoparticles for thrombolytic therapy: Preparation and pharmacodynamics in vivo, J. Thromb. Thrombolysis. 36 (2013) 458-468. https://doi.org/10.1007/s11239-013-0951-7.
[24] J. Liao, X. Ren, B. Yang, H. Li, Y. Zhang, Z. Yin, Targeted thrombolysis by using c-RGD-modified N,N,N-Trimethyl Chitosan nanoparticles loaded with lumbrokinase, Drug Dev. Ind. Pharm. 45 (2019) 88-95. https://doi.org/10.1080/03639045.2018.1522324.
[25] S. R. Van Tomme, W. E. Hennink, Biodegradable dextran hydrogels for protein delivery applications, Expert Rev. Med. Devices. 4 (2007) 147-164. https://doi.org/10.1586/17434440.4.2.147.
[26] J. R. McCarthy, I. Y. Sazonova, S. S. Erdem, T. Hara, B. D. Thompson, P. Patel, I. Botnaru, C. P. Lin, G. L. Reed, R. Weissleder, F. A. Jaffer, Multifunctional nanoagent for thrombus-targeted fibrinolytic therapy, Nanomedicine (Lond). 7 (2012) 1017-1028. https://doi.org/10.2217/nnm.11.179.
[27] S. Heid, H. Unterweger, R. Tietze, R. P. Friedrich, B. Weigel, I. Cicha, D. Eberbeck, A. R. Boccaccini, C. Alexiou, S. Lyer, Synthesis and Characterization of Tissue Plasminogen Activator-Functionalized Superparamagnetic Iron Oxide Nanoparticles for Targeted Fibrin Clot Dissolution, Int. J. Mol. Sci. 18 (2017). https://doi.org/10.3390/ijms18091837.
[28] M. Juenet, R. Aid-Launais, B. Li, A. Berger, J. Aerts, V. Ollivier, A. Nicoletti, D. Letourneur, C. Chauvierre, Thrombolytic therapy based on fucoidan-functionalized polymer nanoparticles targeting P-selectin, Biomaterials. 156 (2018) 204-216. https://doi.org/10.1016/j.biomaterials.2017.11.047.
[29] J. M. Lec, Z.-U. Shin, G. T. Mavlonov, I. Y. Abdurakhmonov, T.-H. Yi, Solid-Phase Colorimetric Method for the Quantification of Fucoidan, Appl. Biochem. Biotechnol. 168 (2012) 1019-1024. https://doi.org/10.1007/s12010-012-9837-y.
[30] S. Petersen, A. Fahr, H. Bunjes, Flow cytometry as a new approach to investigate drug transfer between lipid particles, Mol. Pharm. 7 (2010) 350-363. https://doi.org/10.1021/mp900130s.
[31] C. Orset, R. Macrez, A. R. Young, D. Panthou, E. Angles-cano, E. Maubert, V. Agin, D. Vivien, Mouse Model of In Situ Thromboembolic Stroke and Reperfusion, Stroke. 38 (2007) 2771-2778. https://doi.org/10.1161/STROKEAHA.107.487520.
[32] T. E. Furia, CRC Handbook of Food Additives, Second Edi, CRC Press, 1973.
[33] C. Chauvierre, R. Aid-Launais, J. Aerts, M. Maire, L. Chollet, L. Rolland, R. Bonaf, S. Rossi, S. Bussi, C. Cabella, D. Laszlo, T. Fülöp, J. Szebeni, Y. Chahid, K. H. Zheng, E. S. G. Stroes, D. Le Guludec, F. Rouzet, D. Letourneur, Pharmaceutical Development and Safety Evaluation of a GMP-Grade Fucoidan for Molecular Diagnosis of Cardiovascular Diseases, Mar. Drugs. 17 (2019) 1-17. https://doi.org/https://doi.org/10.3390/md17120699.
[34] F. Rouzet, L. Bachelet-Violette, J.-M. Alsac, M. Suzuki, A. Meulemans, L. Louedec, A. Petiet, M. Jandrot-Perrus, F. Chaubet, J.-B. Michel, D. Le Guludec, D. Letourneur, Radiolabeled fucoidan as a P-selectin targeting agent for in vivo imaging of platelet-rich thrombus and endothelial activation., J. Nucl. Med. 52 (2011) 1433-1440. https://doi.org/10.2967/jnumed.110.085852.
[35] K. H. Zheng, Y. Kaiser, E. Pocl, H. Verberne, J. Aerts, F. Rouzet, E. Stroes, D. Letourneur, C. Chauvierre, 99Mtc-Fucoidan As Diagnostic Agent For P-Selectin Imaging: First-In-Human Evaluation (Phase I), Atherosclerosis. 287 (2019) c143. https://doi.org/10.1016/j.atherosclerosis.2019.06.425.
[36] C. I. Jones, D. A. Payne, P. D. Hayes, A. R. Naylor, P. R. F. Bell, M. M. Thompson, A. H. Goodall, The antithrombotic effect of dextran-40 in man is due to enhanced fibrinolysis in vivo, J. Vasc. Surg. 48 (2008) 715-722. https://doi.org/10.1016/j.jvs.2008.04.008.
[37] E. M. Balboa, E. Conde, A. Moure, E. Falqué, H. Domínguez, In vitro antioxidant properties of crude extracts and compounds from brown algae, Food Chem. 138 (2013) 1764-1785. https://doi.org/10.1016/j.foodchem.2012.11.026.
[38] Y. Choi, S. K. Min, R. Usoltseva, A. Silchenko, T. Zvyagintseva, S. Ermakova, J. K. Kim, Thrombolytic fucoidans inhibit the tPA-PAI1 complex, indicating activation of plasma tissue-type plasminogen activator is a mechanism of fucoidan-mediated thrombolysis in a mouse thrombosis model, Thromb. Res. 161 (2018) 22-25. https://doi.org/10.1016/j.thromres.2017.11.015.
[39] F. Wolf, K. Kochler, H. P. Schuchmann, Stabilization of water droplets in oil with PGPR for use in oral and dermal applications, J. Food Process Eng. 36 (2013) 276-283. https://doi.org/10.1111/j.1745-4530.2012.00688.x.
[40] K. S. Masters, D. N. Shah, L. A. Leinwand, K. S. Anseth, Crosslinked hyaluronan scaffolds as a biologically active carrier for valvular interstitial cells, Biomaterials. 26 (2005) 2517-2525. https://doi.org/10.1016/j.biomaterials.2004.07.018.
[41] J. Matuszak, J. Baumgartner, J. Zaloga, M. Juenet, A. E. da Silva, D. Franke, G. Almer, I. Texier, D. Faivre, J. M. Metselaar, F. P. Navarro, C. Chauvierre, R. Prassl, L. Dézsi, R. Urbanics, C. Alexiou, H. Mangge, J. Szebeni, D. Letourneur, I. Cicha, Nanoparticles for intravascular applications: physicochemical characterization and cytotoxicity testing, Nanomedicine. 11 (2016) 597-616. https://doi.org/10.2217/nnm.15.216.
[42] M. Weber, H. Steinle, S. Golombek, L. Hann, C. Schlensak, H. P. Wendel, M. Avci-Adali, Blood-Contacting Biomaterials: In Vitro Evaluation of the Hemocompatibility, Front. Bioeng. Biotechnol. 6 (2018). https://doi.org/10.3389/fbioc.2018.00099.
[43] ISO 10993-4:2017, Biol. Eval. Med. Devices—Part 4 Sel. Tests Interact. with Blood. (2017). https://www.iso.org/standard/63448.html.
[44] B. Vaidya, G. P. Agrawal, S. P. Vyas, Platelets directed liposomes for the delivery of streptokinase: Development and characterization, Eur. J. Pharm. Sci. 44 (2011) 589-594. https://doi.org/10.1016/j.cjps.2011.10.004.
[45] N. Zhang, C. Li, D. Zhou, C. Ding. Y. Jin, Q. Tian, X. Meng. K. Pu, Y. Zhu, Cyclic RGD functionalized liposomes encapsulating urokinase for thrombolysis, Acta Biomater. (2018). https://doi.org/10.1016/j.actbio.2018.01.038.
[46] K. Ley, The role of selectins in inflammation and disease, Trends Mol. Med. 9 (2003) 263-268. https://doi.org/10.1016/S1471-4914(03)00071-6.
[47] J.-H. Kim, J.-Y. Yoon, Protein adsorption on polymer particles, in: Encycl. Surf. Colloid Sci., 2002: pp. 4373-4381. https://doi.org/10.1002/jbm.820210202.
[48] I. Politis, L. Wang. J. D. Turner, B. K. Tsang, Changes in Tissue-Type Plasminogen Activator-Like and Plasminogen Activator Inhibitor Activities in Granulosa and Theca Layers during Ovarian Follicle Development in the Domestic Hen1, Biol. Reprod. 42 (1990) 747-754. https://doi.org/10.1095/biolreprod42.5.747.
[49] M. Di Marco, K. A. Razak, A. A. Aziz, C. Devaux, E. Borghi, L. Levy, C. Sadun, Overview of the main methods used to combine proteins with systems: absorption, bioconjugation, and encapsulation, Int. J. Nanomedicine. 5 (2010) 37-49.
[50] V. Wintgens, C. Lorthioir, P. Dubot, B. Sébille, C. Amiel, Cyclodextrin/dextran based hydrogels prepared by cross-linking with sodium trimetaphosphate, Carbohydr. Polym. 132 (2015) 80-88. https://doi.org/10.1016/j.carbpol.2015.06.038.
[51] R.P. Friedrich, J. Zaloga, E. Schreiber, I.Y. Tóth, E. Tombácz, S. Lyer, C. Alexiou, Tissue Plasminogen Activator Binding to Superparamagnetic Iron Oxide Nanoparticle-Covalent Versus Adsorptive Approach, Nanoscale Res. Lett. 11 (2016) 1-11. https://doi.org/10.1186/s11671-016-1521-7.
[52] L. Arnfast, C.G. Madsen, L. Jorgensen, S. Baldursdottir, Design and processing of nanogels as delivery nanosystems for peptides and proteins, Ther. Deliv. 5 (2014) 691-708. https://doi.org/10.4155/tde.14.38.
[53] Z.M. Ruggeri, Platelets in atherothrombosis, Nat. Med. 8 (2002) 1227-1234. https://doi.org/10.4065/81.1.59.
[54] C. Orset, B. Haclewyn, S.M. Allan, S. Ansar, F. Campos, T.H. Cho, A. Durand, M. El Amki, M. Fatar, I. Garcia-Yébenes, M. Gauberti, S. Grudzenski, I. Lizasoain, E. Lo, R. Macrez, I. Margaill, S. Maysami, S. Meairs, N. Nighoghossian, J. Orbe, J.A. Paramo, J.J. Parienti, N.J. Rothwell, M. Rubio, C. Wacber, A.R. Young, E. Touzé, D. Vivien, Efficacy of Alteplase in a Mouse Model of Acute Ischemic Stroke: A Retrospective Pooled Analysis, Stroke. 47 (2016) 1312-1318. https://doi.org/10.1161/STROKEAHA.116.012238.
[55] S.M. De Lizarrondo, C. Gakuba, B.A. Herbig, Y. Repessé, C. Ali, C. V. Denis, P.J. Lenting. E. Touze, S.L. Diamond, D. Vivien, M. Gauberti, Potent thrombolytic effect of N-acetylcysteine on arterial thrombi, Circulation. 136 (2017) 646-660. https://doi.org/10.1161/CIRCULATIONAHA.117.027290.
[56] H.R. Lijnen, B. Van Hoef, V. Beelen, D. Collen, Characterization of the Murine Plasma Fibrinolytic System, Eur. J. Biochem. 224 (1994) 863-871. https://doi.org/10.1111/j.1432-1033.1994.00863.x.
[57] J. Hu, S. Huang. L. Zhu, W. Huang. Y. Zhao, K. Jin, Q. Zhuge, Tissue Plasminogen Activator-Porous Magnetic Microrods for Targeted Thrombolytic Therapy after Ischemic Stroke, ACS Appl. Mater. Interfaces. 10 (2018) 32988-32997. https://doi.org/10.1021/acsami.8b09423.
[58] L. Huang, J. Wang, S. Huang, F. Siaw-Debrah, M. Nyanzu, Q. Zhuge, Polyacrylic acid-coated nanoparticles loaded with recombinant tissue plasminogen activator for the treatment of mice with ischemic stroke, Biochem. Biophys. Res. Commun. 516 (2019) 565-570. https://doi.org/10.1016/j.bbrc.2019.06.079.
[59] T. Mei, A. Kim, L. B. Vong. A. Marushima, S. Puentes, Y. Matsumaru, A. Matsumura, Y. Nagasaki, Encapsulation of tissue plasminogen activator in pH-sensitive self-assembled antioxidant nanoparticles for ischemic stroke treatment-Synergistic effect of thrombolysis and antioxidant, Biomaterials. 215 (2019) 1-12. https://doi.org/10.1016/j.biomaterials.2019.05.020.
[60] R. Brouns, P. P. De Deyn, The complexity of neurobiological processes in acute ischemic stroke, Clin. Neurol. Neurosurg. (2009) 111 483-495. https://doi.org/10.1016/j.clineuro.2009.04.001.
[61] H. Kim, J. H. Ahn, M. Song, D. W. Kim, T. K. Lec, J. C. Lec, Y. M. Kim, J. D. Kim, J. H. Cho, I. K. Hwang. B. C. Yan, M. H. Won, J. H. Park, Pretreated fucoidan confers neuroprotection against transient global cerebral ischemic injury in the gerbil hippocampal CA1 area via reducing of glial cell activation and oxidative stress, Biomed. Pharmacother. 109 (2019) 1718-1727. https://doi.org/10.1016/j.biopha.2018.11.015.
[62] D. Vivien, M. Gauberti, A. Montagne, G. Defer, E. Touzé, Impact of tissue plasminogen activator on the neurovascular unit: From clinical data to experimental evidence, J. Cereb. Blood Flow Metab. 31 2119-2134. (2011) https://doi.org/10.1038/jcbfm.2011.127.
[63] R. P. Friedrich, J. Zaloga, E. Schreiber, I. Y. Tóth, E. Tombácz, S. Lyer, C. Alexiou, Tissue Plasminogen Activator Binding to Superparamagnetic Iron Oxide Nanoparticle-Covalent Versus Adsorptive Approach, Nanoscale Res. Lett. 11 (2016) 1-11. https://doi.org/10.1186/s11671-016-1521-7.
[64] S. Yokoyama, H. Ikeda, N. Haramaki, H. Yasukawa, T. Murohara, T. Imaizumi, Platelet P-selectin plays an important role in arterial thrombogenesis by forming large stable platelet-leukocyte aggregates, J. Am. Coll. Cardiol. 45 (2005) 1280-1286. https://doi.org/10.1016/j.jacc.2004.12.071.
[65] S. R. Barthel, J. D. Gavino, L. Descheny, C. J. Dimitroff, Targeting selectins and selectin ligands in inflammation and cancer, Expert Opin. Ther. Targets. 11 (2007) 1473-1491. https://doi.org/10.1517/14728222.11.11.1473.
[66] Y. Shamay. M. Elkabets, H. Li, J. Shah, S. Brook, F. Wang, K. Adler, E. Baut, M. Scaltriti, P. V Jena, E. E. Gardner, J. T. Poirier, C. M. Rudin, J. Baselga, A. Haimovitz-Friedman, D. A. Heller, P-selectin is a nanotherapeutic delivery target in the tumor microenvironment, Sci. Transl. Med. (2016). https://doi.org/10.1126/scitranslmed.aaf7374.

1. A cross-linked polysaccharide particle comprising fucoidan and loaded by adsorption with t-PA.

2. The cross-linked polysaccharide particle of claim 1 wherein the polysaccharide is selected from the group consisting of dextran, pullulan, carboxymethyl dextran, agar, alginic acid, hyaluronic acid, inulin, heparin, chitosan and mixtures thereof.

3. The cross-linked polysaccharide particle of claim 1 wherein the polysaccharide is dextran.

4. The cross-linked polysaccharide particle of claim 1 wherein the fucoidan has an average molecular weight ranging from 2 kDa to 100 kDa.

5. A method of obtaining the cross-linked polysaccharide particle of claim 1 comprising the steps of
a) preparing an alkaline aqueous solution comprising an amount of at least one polysaccharide, the fucoidan and a cross linking agent;
b) dispersing said alkaline aqueous solution into a hydrophobic phase in order to obtain a w/o emulsion; and
c) transforming the w/o emulsion into particles by placing said w/o emulsion at a temperature from about 4° C. to about 80° C. for a sufficient time to allow the cross-linking of said at least one polysaccharide and fucoidan,
d) loading the t-PA into the particles obtained at step c).

6. The method of claim 5 wherein the cross-linking agent is selected from the group consisting of trisodium trimetaphosphate (STMP), phosphorus oxychloride (POC13), epichlorohydrin, formaldehydes, carbodiimides, and glutaraldehydes.

7. The method of claim 5 wherein the cross-linking agent is trisodium trimetaphosphate (STMP).

8. The method of claim 5 wherein hydrophobic phase of step b) is sunflower oil.

9. The method of claim 5 wherein step b) is carried out in the presence of a surfactant.

10. The method of claim 9 wherein the surfactant is selected from the group consisting of polyglycerol polyricinoleate and PEG-30 dipolyhydroxystearate.

11. The method of claim 5 wherein step b) is carried out in the presence of an osmotic agent.

12. The method of claim 11 wherein the osmotic agent is selected from the group consisting of salts, sugars, volatile solutes and mixtures thereof.

13. The method of claim 5 wherein the loading of t-PA into the particles obtained at step c) is carried out by adsorption by mixing the particles with a solution comprising of t-PA.

14. A method of treating a thrombotic disease or disorder in a patient in need thereof comprising administering to the patient a therapeutically effective amount the cross-linked polysaccharide particle of claim 1.

15. A pharmaceutical composition comprising of the cross-linked polysaccharide particle of claim 1.

16. The method of claim 12 wherein
the salt is NaCl, MgCl₂, or KN0₃, and/or
the sugar is sucrose, glucose or fructose, and/or
the volatile solute is SO₂.
